(19) United States
(12) Patent Application Publication
(10) Pub. No.: US 2026/0020910 A1
(43) Pub. Date: Jan. 22, 2026

(54) SYSTEMS AND METHODS FOR PLANNING A PATELLA REPLACEMENT PROCEDURE

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Ruxandra C. Marinescu Tanasoca, Memphis, TN (US); Ashley A. Roakes, Memphis, TN (US); Elizabeth A. Duxbury, Kernville, CA (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/099,949

(22) PCT Filed: Oct. 25, 2023
(86) PCT No.: PCT/US23/35888

§ 371 Date: Jan. 30, 2025

Related U.S. Application Data
(60) Provisional application No. 63/419,471, filed on Oct. 26, 2022.

(51) Int. Cl. A61B 34/10; A61B 34/00

(52) U.S. Cl. CPC A61B 34/10; A61B 34/25; A61B 2034/102; A61B 2034/105; A61B 2034/107; A61B 2034/108

(57) ABSTRACT

Disclosed herein are systems and methods for planning a knee arthroplasty procedure. A method may include registration of patient anatomy, including specific registration of the patella; anatomy modelling based on the registration; automated generation of anatomical landmarks; morphological characterization; risk classification; and a planning optimization process based on the morphological characterization and the risk classification. The risk classification may include a determination of potential patellar complications based on patient characteristics, such as patient demographic information and/or patient anatomical information compared with historical information of patient outcomes. The planning optimization process may determine knee implant components and/or the configuration thereof within the patient based on the patient risk classification.

### CROSS-REFERENCE TO RELATED APPLICATION

This application claims the benefit of U.S. Provisional Patent Application Ser. No. 63/419,471, filed Oct. 26, 2022, and titled “Systems and Methods for Planning a Patella Replacement Procedure,” the entire contents of which application are hereby incorporated by reference in their entirety.

### TECHNICAL FIELD

The present disclosure generally relates to methods, systems, and apparatuses related to a computer-assisted surgical system that includes various hardware and software components that work together to enhance surgical workflows. More specifically, the present disclosure relates to methods, systems, and apparatuses for determining patella characteristics of a patient and optimizing patellar implant selection and placement for a particular patient to achieve successful patient outcomes in a knee arthroplasty procedure.

### BACKGROUND

Patella and patella tracking problems account for a substantive number of poor total knee arthroplasty (TKA) outcomes in patients. For example, anterior knee pain after TKA is a typical post-surgery patellofemoral complication that may be attributed to issues with a native or implant patella.

Currently, robot-or computer-assisted TKA may be used for the preparation of the femur and tibia, for example, optimizing implant placement, leg alignment, soft tissue balancing, range of motion assessment, etc. Although patella mal-positioning can result in patellar maltracking and, ultimately, pain and other significant complications, the patella is currently not adequately addressed in conventional computer-assisted TKA.

For conventional TKA, including computer-assisted TKA, the prominent surgical technique for patellar replacement is a free-hand technique. For example, before making the patellar cut, the surgeon measures the native or pre-cut patellar thickness with a surgical caliper and then makes the cut or free hands the patella. After the resection, the surgeon measures the post-cut patella thickness. The goal is to restore native patellar thickness, which may be approximately 25 millimeters (mm) for a male patella and 22 mm for a female patella. Surgeons may resect the patella just under the articular surface and not cut the patella to a certain minimum thickness, such as less than about 12-15 mm.

Using conventional techniques, the surgeon assumes that the patella, and patellar tracking, will be adequate post-surgery if the femoral and tibial components are placed correctly. However, misplacement of the patella can lead to various post-operative difficulties, including patella fracture, patellar clunk syndrome, patellofemoral instability, patellar maltracking (i.e., patellar tracking disorder), patella alta, patella baja, and patellar malrotation. The conventional free hand technique does not target or account for the intricate biomechanics of the patellofemoral joint.

For example, during a TKA, surgeons are required to simultaneously process complex information and make a plurality of difficult decisions in the operating room in rapid succession. A patellar cut must be planned and executed with an adequate cut thickness and an appropriate implant size, and a design must be selected for the patellar component. Additionally, a position and orientation of the patellar component must be selected in conjunction with femoral and tibial component positions and orientations. Furthermore, the biomechanics of the patellofemoral joint may be determined in an effort to restore the normal function of the knee joint. Given the complexity of the patellofemoral anatomy and biomechanics, there is a high risk of patellofemoral complications because surgeons are required to make these various planning decisions intraoperatively with minimal available information and tools.

Accordingly, there remains a need for improved techniques for patella replacement planning during a knee arthroplasty procedure. It is with this in mind that the present disclosure is provided.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

Disclosed herein are improved systems and methods for planning a knee arthroplasty procedure, including determining optimal implant components and/or the configuration or alignment of the implant components. In any preceding or subsequent example, the improved systems and methods may operate to perform a surgical process that includes a patella configuration process to determine an optimal patella implant device and/or femur implant device to correspond with the patella and/or patient characteristics. In any preceding or subsequent example, the surgical process may include determining a risk classification of a patient based on patient characteristics, including, without limitation, patient demographic information and anatomical information. The risk classification may be configured to facilitate the determination of the optimal knee implant configuration, for instance, optimal patella implant device and/or femur implant device and configuration/alignment of the patella implant device and/or femur implant device within the patient.

A method of planning a knee arthroplasty procedure of a patient is provided. The knee arthroplasty procedure may include the planning and replacement of a patella of the knee. The method may include receiving input related to an anatomy of the patient and patient characteristics; determining one or more biomechanical measurements of a patellofemoral joint of the knee based on the input; generating a three-dimensional (3D) model of the anatomy based on the input, including a patella model; determining anatomical landmarks of portions of the knee, determining a risk classification of the patient; performing one or more biomechanical simulations based on the risk classification; optimizing a position and an orientation of the implant based on the one or more biomechanical simulations; and determining patient-specific planning information related to the optimized position and the optimized orientation.

In any preceding or subsequent example, a method may include registration of patient anatomy, including specific registration of the patella; anatomy modelling based on the registration; automated generation of anatomical landmarks; morphological characterization; risk classification; and a planning optimization process based on the morphological characterization and the risk classification.

In any preceding or subsequent example, the planning optimization process may be operative to determine knee implant components optimized for a patient and/or the optimized placement, configuration, alignment, and/or the like of the knee implant components for the patient. In any preceding or subsequent example, the knee implant components may include a native patella. In any preceding or subsequent example, the knee implant components may include a patella implant. In any preceding or subsequent example, the knee implant components may include a femur implant component.

In any preceding or subsequent example, the registration of patient anatomy may include measuring, gathering, or otherwise determining information associated with a femur, tibia, patella, surface mapping, range of motion (ROM) collection, and/or the like. In any preceding or subsequent example, registration may include a surface mapping step for the femoral and tibial condyles, for instance, to create a virtual 3D representation of patient anatomy, generated from the collected points. In any preceding or subsequent example, registration may include a specific patella registration step. In any preceding or subsequent example, a device may be associated with the patella to facilitate registration. For instance, a fixture device may be fixed in place on the patella, for example, with bone pins in the patella, clamps, (temporary) adhesive, and/or the like.

In any preceding or subsequent example, anatomy modelling may be or may include generating 3D models of patient anatomy including, without limitation, a femur, a tibia, a patella, ligament attachments, axes, and/or portions thereof.

In any preceding or subsequent example, the generation of anatomical landmarks may include using the generated 3D models to perform a more detailed automatic landmarking process. In any preceding or subsequent example, landmarking points may be generated for a patella. Non-limiting landmarking points for the patella may include width points (medial+lateral), height points (superior+inferior), depth/thickness points (anterior+posterior), inferior articular point, articular perimeter points, posterior ridge points, centroid of the patella, patella neutral point (for instance, aligned with posterior ridge and facet midpoint line), and/or the like.

In any preceding or subsequent example, the morphological characterization may include characterization of the patella, femur (e.g., femoral trochlear groove), patellar tracking, and/or the like. For instance, patella and femoral trochlear groove measurements may be automatically calculated using generated landmarks. From a generated 3D model of the patella, a full morphological characterization can be performed by calculating several key patella measurements including, without limitation, patella width, maximum patella height, patella thickness, lateral facet width, medial facet width, articular surface height, quadriceps angle, and/or posterior patellar angle.

In any preceding or subsequent example, characterization of the femoral trochlear groove may include determining femoral trochlear groove primary axes, femur widths, sulcus points, sulcus angles, trochlear groove depth, facet asymmetry, a lateral trochlear inclination angle, Tibial Tubercle-Trochlear Groove (TT-TG) offset, femoral trochlear groove classification, J-Curves, axial groove angle, femoral trochlear groove medial-lateral position, an epicondylar midpoint, and/or a femoral trochlear groove curvature.

In any preceding or subsequent example, the morphological characterization may include an implant trochlear groove characterization process. In any preceding or subsequent example, determining the implant trochlear groove characteristics may include generating an implanted sulcus model and determining the measurements, calculations, and/or the like associated with the corresponding femoral component.

In any preceding or subsequent example, patient information may be used to determine risk information for a patient. In any preceding or subsequent example, the risk information may be or may include a risk classification and/or risk profile associated with potential outcomes of a knee arthroplasty procedure. In any preceding or subsequent example, the risk information may be used to predict an outcome of a knee arthroplasty procedure (or a portion or characteristic thereof) for a patient. In any preceding or subsequent example, the patient characteristics may include any characteristic that may be used to determine a patient outcome of a knee arthroplasty procedure, including, without limitation, age, gender, ethnicity, behavior, medical diagnostic or imaging information, and/or the like.

In any preceding or subsequent example, a risk level, score, classification, group, indicator, and/or the like may be determined and assigned to each patient, based on the calculated patient risk profile.

In any preceding or subsequent example, based on the patient geometric or anatomical data in combination with intraoperative data (for instance, passive flexion kinematics, gap data from a soft tissue tensioner, and/or the like) and the patient risk classification, the implant optimization process may be operative to determine one or more optimal implants, optimal positioning of the implants, a surgical plan for preparing the patient bones, and/or implanting the implant components. In any preceding or subsequent example, for instance, the selection and/or configuration of knee implant components may be determined to address patient-specific risk factors in order to directly reduce or even eliminate potential post-operative issues that are personal to a patient's unique anatomy and risk factors, for instance, based on the risk classification.

Examples described in the present disclosure provide numerous advantages over conventional systems and methods. In one non-limiting example advantage, risk stratification may allow better surgical decision-making, set realistic patient expectations, make better use of resources, identification of high-risk groups for complications, anticipate complications, predict a clinical outcome, and help the surgeon to proactively plan the most appropriate treatment for that patient. In another non-limiting example, proactively identifying a potential high-risk group or level for a certain patient may give a direction to the surgeon in terms of implant choices, what implant would be best for that patient, modifying the level of constraint, necessary implants and instruments, surgical approaches, implant positioning, and/or the like. In a further non-limiting example, capturing and characterizing patellar behavior may improve clinical outcomes and reduce adverse effects related to patellar mal-positioning and maltracking. Using automated tools to perform the characterization, analysis, and performance recommendations may enhance outcomes without any additional time or patient impact. Conventional systems may provide rudimentary patella modelling, for instance, 3D and/or 2D patella models. However, such conventional patella models are not sufficient to provide meaningful insight for a surgical team; rather, the conventional patella models may merely provide a basic, non-personalized patella that is not formed, processed, analyzed, etc. using computational models configured according to any preceding or subsequent example. Conventional patella models are insufficient to provide for patella risk assessment, surgical planning, implant component selection and/or configuration, and/or the like contrary to patella models and surgical systems according to any preceding or subsequent example.

In many examples, one or more of the components described herein may be implemented as a set of rules that improve computer-related technology by allowing a function not previously performable by a computer that enables an improved technological result to be achieved. For example, components may facilitate a computer to generate the technological result of determining a patella risk classification for a patient based on patient physiological information and machine learning processes. Components may facilitate a computer to generate the technological result of providing an optimized implant device that directly addresses patient anatomy in based on the risk classification in combination with the unique risk factors associated with the patient. This technological result may allow a computing device, programmed to operate according to example processes described in the present disclosure, to provide the functionality of determining knee implant components and the configuration of knee implant components, including, specifically, the patella and associated trochlear groove of the femur (or femoral component) to achieve improved surgical outcomes for the patient. This technological result may allow a computing device, programmed to operate according to example processes described in the present disclosure, to provide the functionality of determining a surgical plan that addresses the risks associated with the determined risk classification to reduce or even eliminate the potential issues affecting a successful surgical outcome.

In any preceding or subsequent example, a computer-implemented method for planning a knee arthroplasty procedure may include, via a processor of a computing device: receiving anatomical information of a knee joint, the anatomical information comprising patella information of a femur of a patient and femoral information of a femur of the patient, generating three-dimensional (3D) models of at least one portion of the knee joint based on the anatomical information, the 3D models comprising a patella model and a femoral model; characterizing a knee morphology of the knee joint, the knee morphology comprising a patella morphology of the patella and a femur morphology of the femur based on the 3D models; determining a patella risk classification based on the knee morphology, the patella risk classification indicating a risk of a patella complication of the knee arthroplasty procedure.

In any preceding or subsequent example of the computer-implemented method, the method may include determining patella landmarks based on the patella model, wherein the knee morphology is determined based on the patella landmarks.

In any preceding or subsequent example of the computer-implemented method, the patella landmarks may include one or more of width points, height points, depth/thickness points, inferior articular point, articular perimeter points, posterior ridge points, centroid of the patella, or patella neutral point.

In any preceding or subsequent example of the computer-implemented method, the femur of the knee joint may include a femoral implant, wherein the femoral information may include determined information for the femoral implant.

In any preceding or subsequent example of the computer-implemented method, the femur morphology may include an implant trochlear groove morphology.

In any preceding or subsequent example of the computer-implemented method, the implant trochlear groove morphology may be determined based on a sulcus model associated with the femoral component.

In any preceding or subsequent example of the computer-implemented method, the patella risk classification may include at least one of a risk of maltracking of the patella, a risk of post-operative knee pain, or patellofemoral instability.

In any preceding or subsequent example of the computer-implemented method, the patella risk classification may be based on one or more risk factors comprising at least one of a Wiberg index, patella size, pre-operative patellar shift, or femoral sulcus groove depth.

In any preceding or subsequent example of the computer-implemented method, the method may include determining a surgical plan based on the patella risk classification, the surgical plan configured to address at least one complication associated with the patella risk classification.

In any preceding or subsequent example of the computer-implemented method, the surgical plan may include a determination of an optimal femoral component for the patella risk classification.

In any preceding or subsequent example of the computer-implemented method, the surgical plan may include a determination of an optimal femoral component placement for the patella risk classification.

In any preceding or subsequent example of the computer-implemented method, the surgical plan may include a determination of an optimal femoral preparation factor for the patella risk classification, the femoral preparation factor comprising a resection depth.

In any preceding or subsequent example, a computer-assisted surgical system may include at least one computing device which may include a display device; processing circuitry; and a memory coupled to the processing circuitry, the memory may include instructions that, when executed by the processing circuitry, cause the processing circuitry to receive anatomical information of a knee joint, the anatomical information may include patella information of a femur of a patient and femoral information of a femur of the patient, generate three-dimensional (3D) models of at least one portion of the knee joint based on the anatomical information, the 3D models may include a patella model and a femoral model; characterize a knee morphology of the knee joint, the knee morphology may include a patella morphology of the patella and a femur morphology of the femur based on the 3D models; and determine a patella risk classification based on the knee morphology, the patella risk classification indicating a risk of a patella complication of the knee arthroplasty procedure.

In any preceding or subsequent example of the system, the instructions, when executed by the processing circuitry, to cause the processing circuitry to determine patella landmarks based on the patella model, wherein the knee morphology is determined based on the patella landmarks.

In any preceding or subsequent example of the system, the patella landmarks may include one or more of width points, height points, depth/thickness points, inferior articular point, articular perimeter points, posterior ridge points, centroid of the patella, or patella neutral point.

In any preceding or subsequent example of the system, the femur of the knee joint may include a femoral implant, wherein the femoral information comprises determined information for the femoral implant.

In any preceding or subsequent example of the system, the femur morphology may include an implant trochlear groove morphology.

In any preceding or subsequent example of the system, the implant trochlear groove morphology determined based on a sulcus model associated with the femoral component.

In any preceding or subsequent example of the system, the patella risk classification may include at least one of a risk of maltracking of the patella, a risk of post-operative knee pain, or patellofemoral instability.

In any preceding or subsequent example of the system, the patella risk classification based on one or more risk factors may include at least one of a Wiberg index, patella size, pre-operative patellar shift, or femoral sulcus groove depth.

In any preceding or subsequent example of the system, the instructions, when executed by the processing circuitry, to cause the processing circuitry to determine a surgical plan based on the patella risk classification, the surgical plan configured to address at least one complication associated with the patella risk classification.

In any preceding or subsequent example of the system, the surgical plan may include a determination of an optimal femoral component for the patella risk classification.

In any preceding or subsequent example of the system, the surgical plan may include a determination of an optimal femoral component placement for the patella risk classification.

In any preceding or subsequent example of the system, the surgical plan may include a determination of an optimal femoral preparation factor for the patella risk classification, the femoral preparation factor may include a resection depth.

Further features and advantages of at least some of the examples described in the present disclosure, as well as the structure and operation of any preceding or subsequent example of the present disclosure, are described in detail below with reference to the accompanying drawings.

### BRIEF DESCRIPTION OF THE DRAWINGS

By way of example, specific examples of the disclosed device will now be described, with reference to the accompanying drawings, in which:

FIG. 1 depicts an operating theatre including an illustrative computer-assisted surgical system (CASS) in accordance with one or more features of the present disclosure;

FIG. 2A depicts illustrative control instructions that a surgical computer provides to other components of a CASS in accordance with one or more features of the present disclosure;

FIG. 2B depicts illustrative control instructions that components of a CASS provide to a surgical computer in accordance with one or more features of the present disclosure;

FIG. 2C depicts an illustrative implementation in which a surgical computer is connected to a surgical data server via a network in accordance with one or more features of the present disclosure;

FIG. 3 depicts an operative patient care system and illustrative data sources in accordance with one or more features of the present disclosure;

FIG. 4A depicts an illustrative flow diagram for determining a pre-operative surgical plan in accordance with one or more features of the present disclosure;

FIG. 4B depicts an illustrative flow diagram for determining an episode of care including pre-operative, intraoperative, and post-operative actions in accordance with one or more features of the present disclosure;

FIGS. 4C-4E depict illustrative graphical user interfaces including images depicting an implant placement in accordance with one or more features of the present disclosure;

FIG. 5 illustrates an example of a surgical workflow in accordance with one or more features of the present disclosure;

FIG. 6A depicts illustrative automatic landmarking points for a femur in accordance with one or more features of the present disclosure;

FIG. 6B depicts illustrative automatic landmarking points for a tibia in accordance with one or more features of the present disclosure;

FIG. 6C depicts illustrative automatic landmarking points for a patella in accordance with one or more features of the present disclosure;

FIG. 7 illustrates an example of a workflow for characterizing a femoral trochlear groove and associated patient anatomy;

FIG. 8A depicts an illustrative femur model showing axes for a femur in accordance with one or more features of the present disclosure;

FIG. 8B depicts illustrative points for determining femoral size of a femur model in accordance with the present disclosure;

FIG. 8C depicts illustrative sulcus points and sulcus lines for a femur model in accordance with the present disclosure;

FIG. 8D depicts an illustrative sulcus angle for a femur model in accordance with the present disclosure;

FIG. 8E depicts an illustrative femoral trochlear groove depth for a femur model in accordance with the present disclosure;

FIG. 8F depicts determining facet asymmetry for a femur model in accordance with the present disclosure;

FIG. 8G depicts an illustrative lateral trochlear inclination angle for a femur model in accordance with the present disclosure;

FIG. 8H depicts an illustrative Tibial Tubercle-Trochlear Groove offset for a femur model in accordance with the present disclosure;

FIG. 8I depicts determining J-Curves for a femur model in accordance with the present disclosure;

FIG. 8J depicts an illustrative axial groove angle and a frontal groove angle for a femur model in accordance with the present disclosure;

FIG. 8K depicts an illustrative mediolateral (ML) position of the femoral trochlear groove for a femur model in accordance with the present disclosure;

FIG. 8L depicts an illustrative trans-epicondylar axis (TEA) midpoint for a femur model in accordance with the present disclosure;

FIG. 8M depicts illustrative femoral trochlear groove curvatures for a femur model in accordance with the present disclosure;

FIG. 8N depicts characterizing a trochlear groove for a femur model of a femur implant device in accordance with the present disclosure;

FIG. 9 depicts an illustrative first operating environment for a planning optimizer process in accordance with the present disclosure;

FIG. 10 depicts an illustrative second operating environment for a planning optimizer process in accordance with the present disclosure;

FIG. 11 depicts an illustrative third operating environment for a planning optimizer process in accordance with the present disclosure; and

FIG. 12 depicts an illustrative operating environment for a surgical planning process in accordance with the present disclosure.

It should be understood that the drawings are not necessarily to scale and that the disclosed examples are sometimes illustrated diagrammatically and in partial views. In certain instances, details which are not necessary for an understanding of the disclosed methods and devices, or which render other details difficult to perceive may have been omitted. It should be further understood that this disclosure is not limited to the particular examples illustrated herein. In the drawings, like numbers refer to like elements throughout unless otherwise noted.

### DETAILED DESCRIPTION

This disclosure is not limited to the particular systems, devices and methods described, as these may vary. The terminology used in the description is for the purpose of describing the particular versions or examples only and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to.”

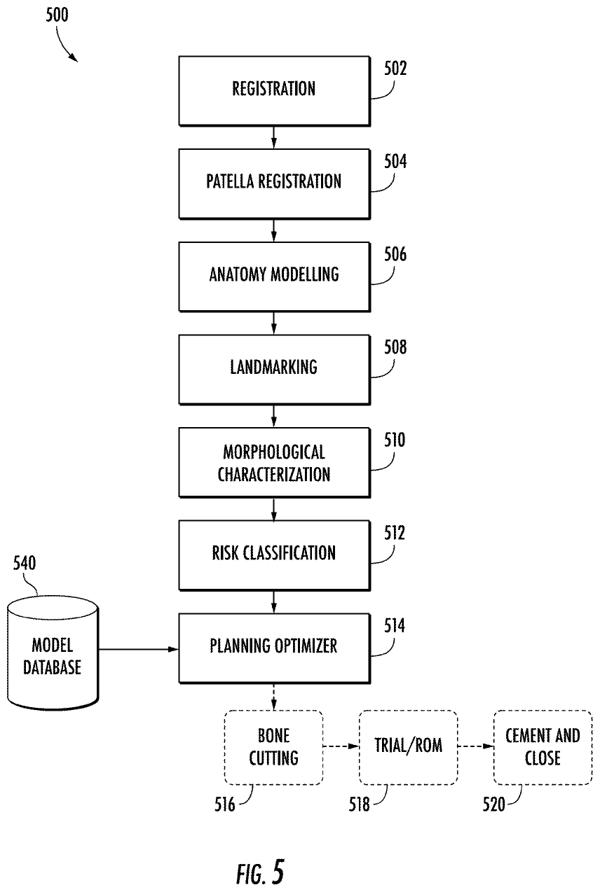

The described technology generally relates to surgical processes, for example, knee arthroplasty procedures including, without limitation, a total knee arthroplasty (TKA) procedure. In any preceding or subsequent example, a surgical process may include a method for optimizing patellofemoral tracking and/or placement of the patellar implant through patient anatomy characterization. A surgical workflow according to any preceding or subsequent example may include registering the patient anatomy (e.g., femur, tibia, and/or patella), generating models and/or anatomical landmarks, patella morphological characterization, femur morphological characterization, classifying the bones, and determining a patient classification, for example, identifying the risk for patellar complications as input into an implant planning optimizer, bone cutting, implant trialing, final implant placement, and/or other surgical steps (see, for example, FIG. 5).

In any preceding or subsequent example, surgical processes may be or may include a surgical workflow for computer-assisted or navigated knee arthroplasty procedures. A surgical workflow may include registering the patient's anatomy intra-operatively in order to create a three-dimensional (3D) representation of the femur, tibia, and patellar anatomy. The patient's anatomy may be automatically landmarked and measured in order to characterize the anatomy, including the patellar anatomy, and classify the femur and patella into classification groups that are associated with risk of patellar complications. After risk classification, a module or tool (for instance, an implant planning optimizer) may provide optimization information to the surgeon, such as risk of maltracking, in order to guide implant placement, for instance, medial-lateral position, resection depth, resection plane, and/or the like and/or to inform implant selection such as shape or size. After implant planning, the surgeon can perform certain surgical steps, such as performing the surgical cuts, placing trials, including a patellar implant trial for capturing range of motion (ROM), collecting post-operative baseline information, confirming implant size, shape, position, and orientation, and/or the like. After trialing, the surgeon may finish the implantation of the remaining components.

There are multiple benefits to computer-assisted surgery, which has led to its widespread adoption and promoted technological advancement. For knee arthroplasty procedures, benefits may include more accurate implant size, position, and orientation, improved range of motion, improved soft tissue balance, reduced risk of injury to soft tissues, reduced outliers, quicker recovery, and/or reduced post-operative pain. Overall, computer-assisted knee arthroplasty may allow for more accurate and precise bone cuts, implant placement, and/or better joint alignment which, ultimately, facilitates improved patient outcomes.

However, although important to the outcome of the surgery, the patella and patellar tracking have not been included in patient-specific or computer-assisted surgery technologies or techniques. For instance, for surgical systems that use patient-specific instrumentation (PSI), such as custom-made bone cutting guides, patient-specific instruments and other smart surgical planning tools usually include only femur and tibia preparation and pre-op alignment, and do not include patellar cutting guides or patella preparation. Even within a computer-assisted surgical setting, knee arthroplasty procedures do not include automated, computer-assisted patellar tracking and patella preparation processes. Rather, the patella is typically resected using a free hand technique.

Although patella mal-positioning can result in patellar maltracking and ultimately pain and other significant complications, the patella is currently not addressed in current computer-assisted TKA. Introducing additional steps in the surgical workflow, to not only capture the patellar anatomy and characterize patellar tracking but also to predict clinical outcomes, is critical in providing a surgeon with a better understanding as to how the patellar tracking can be optimized to reduce the risk of poor outcomes.

Accordingly, any preceding or subsequent example may include technologies and processes for determining patella information to optimize the patellar tracking and the post-operative biomechanics and performance of the patella.

In any preceding or subsequent example, the surgical process may include a patient classification process. In any preceding or subsequent example, the patient classification process may include classifying a patient and/or anatomical portions thereof (e.g., femur, tibia, patella, portions thereof) into a classification group. In any preceding or subsequent example, the classification groups may be or may be associated with a risk classification. In any preceding or subsequent example, the risk classification may be or may include a risk of patellar complications. The risk classification may be or may include a patient risk profile. For example, patient factors (including, for example and without limitation, age, gender, ethnicity, weight, height, body mass index (BMI), clinical characteristics, and radiological findings (patellar height alta/baja, patellar tilt, patellar shift, posterior patellar angle, elongated patellar tendons, trochlear dysplasia, femoral sulcus angle, etc.), and/or the like) can be used to build the patient risk profile. In any preceding or subsequent example, a 3D model of the patella may be generated based upon the pre-operative characterization and the risk classification or profile associated with the patient.

For instance, in any preceding or subsequent example, a risk level or a risk group may be assigned to each patient, based on the calculated patient risk profile. Different risk levels or groups may be determined, provided, or otherwise used depending, for example, on various characteristics, such as the case complexity. Risk stratification according to any preceding or subsequent example may provide multiple advantages over existing systems, including, without limitation, better surgical decision-making, set realistic patient expectations, make better use of resources, identification of high-risk groups for complications, anticipate complications, predict a clinical outcome, and help the surgeon to proactively plan the most appropriate treatment for that patient. In another non-limiting example of a technological advantage, proactively identifying a potential high-risk group or level for a certain patient may provide a direction to the surgeon in terms of implant choices, what implant would be best for that patient, constraint decisions (e.g., increasing/decreasing the level of constraint, what implants and instruments might be needed, what would be the best surgical approach, how best to position those implants, and/or the like).

As a result, surgical processes including patient classification according to any preceding or subsequent example may provide surgeons with improved surgical methods that are more accurate and reduce complexity and cognitive load (particularly during an active surgery), while also improving patient outcomes.

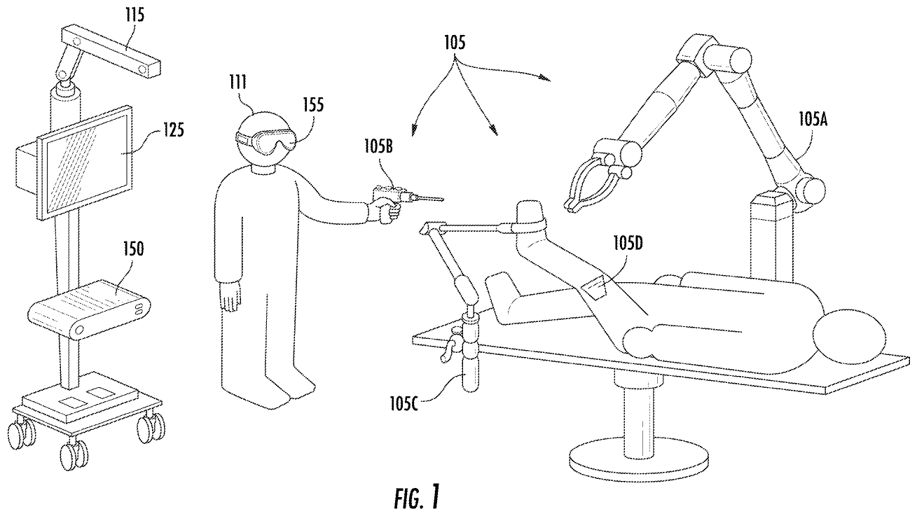

FIG. 1 depicts an example computer-assisted surgical system (CASS) 100 according to any preceding or subsequent example that uses computers, robotics, and imaging technology to aid surgeons in performing orthopedic surgery procedures such as knee arthroplasty (e.g., total knee arthroplasty (TKA)) or total hip arthroplasty (THA). An Effector Platform 105 positions surgical tools relative to a patient during surgery. For example, for a knee surgery, the Effector Platform 105 may include an End Effector 105B that holds surgical tools or instruments during their use. Effector Platform 105 can include a Limb Positioner 105C for positioning the patient's limbs during surgery. Resection Equipment 110 (not shown in FIG. 1) performs bone or tissue resection using, for example, mechanical, ultrasonic, or laser techniques. Effector Platform 105 can also include a cutting guide or jig 105D that is used to guide saws or drills used to resect tissue during surgery. Such cutting guides 105D can be formed integrally as part of the Effector Platform 105 or Robotic Arm 105A, or cutting guides can be separate structures that can be matingly and/or removably attached to the Effector Platform 105 or Robotic Arm 105A.

The Tracking System 115 uses one or more sensors to collect real-time position data that locates the patient's anatomy and surgical instruments. Any suitable tracking system can be used for tracking surgical objects and patient anatomy in the surgical theatre. For example, a combination of infrared (IR) and visible light cameras can be used in an array.

The registration process that registers the CASS 100 to the relevant anatomy of the patient can also involve the use of anatomical landmarks, such as landmarks on a bone or cartilage. For example, the CASS 100 can include a 3D model of the relevant bone or joint and the surgeon can intraoperatively collect data regarding the location of bony landmarks on the patient's actual bone using a probe that is connected to the CASS. Alternatively, the CASS 100 can construct a 3D model of the bone or joint without pre-operative image data by using location data of bony landmarks and the bone surface that are collected by the surgeon using a CASS probe or other means.

A Tissue Navigation System (not shown in FIG. 1) provides the surgeon with intraoperative, real-time visualization for the patient's bone, cartilage, muscle, nervous, and/or vascular tissues surrounding the surgical area.

The Display 125 provides graphical user interfaces (GUIs) that display images collected by the Tissue Navigation System as well other information relevant to the surgery. For example, the Display 125 overlays image information collected from various modalities (e.g., CT, MRI, X-ray, fluorescent, ultrasound, etc.) collected pre-operatively or intra-operatively to give the surgeon various views of the patient's anatomy as well as real-time conditions. Surgical Computer 150 provides control instructions to various components of the CASS 100, collects data from those components, and provides general processing for various data needed during surgery.

Data acquired during the pre-operative phase generally includes all information collected or generated prior to the surgery. Thus, for example, information about the patient may be acquired from a patient intake form or electronic medical record (EMR). Examples of patient information that may be collected include, without limitation, patient demographics, diagnoses, medical histories, progress notes, vital signs, medical history information, allergies, and lab results. The pre-operative data may also include images related to the anatomical area of interest. These images may be captured, for example, using Magnetic Resonance Imaging (MRI), Computed Tomography (CT), X-ray, ultrasound, or any other modality known in the art.

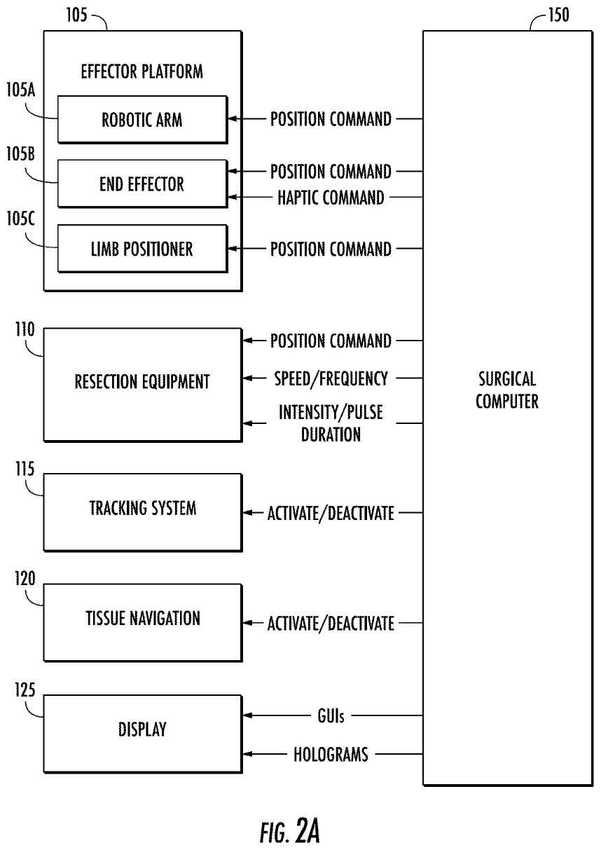

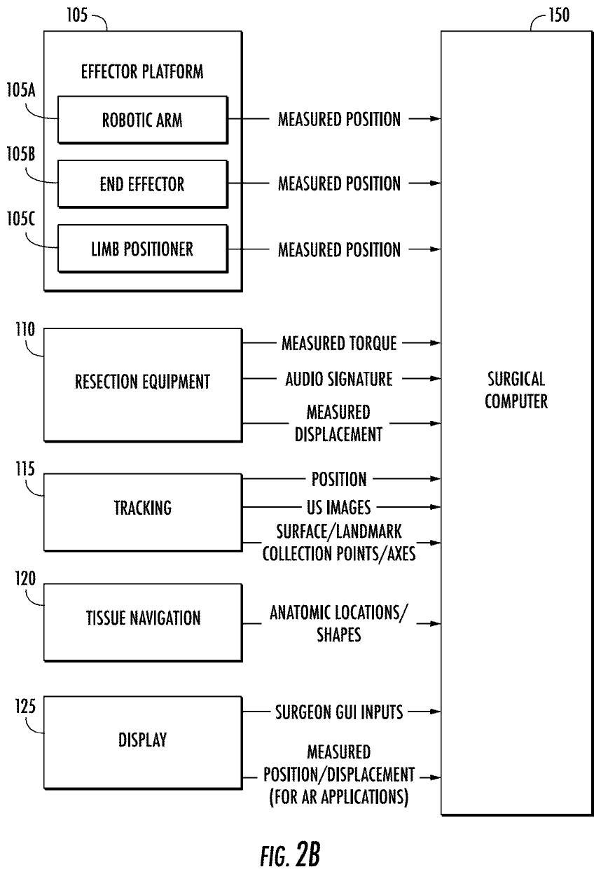

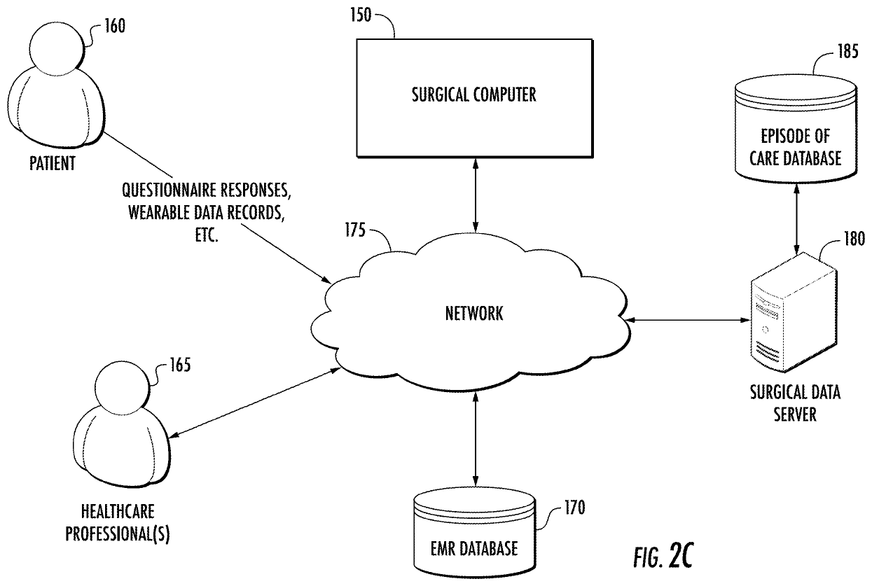

FIGS. 2A and 2B provide examples of data that may be acquired during the intra-operative phase of an episode of care. These examples are based on the various components of the CASS 100 described above with reference to FIG. 1; however, it should be understood that other types of data may be used based on the types of equipment used during surgery and their use. FIG. 2A shows examples of some of the control instructions that the Surgical Computer 150 provides to other components of the CASS 100, according to any preceding or subsequent example.

Resection Equipment 110 is provided with a variety of commands to perform bone or tissue operations. As with the Effector Platform 105, position information may be provided to the Resection Equipment 110 to specify where it should be located when performing resection.

During the registration process, for example, the Display 125 can show a preoperatively constructed 3D bone model and depict the locations of the probe as the surgeon uses the probe to collect locations of anatomical landmarks on the patient. The Display 125 can include information about the surgical target area. For example, in connection with a TKA, the Display 125 can depict the mechanical and anatomical axes of the femur and tibia. The Display 125 can depict varus and valgus angles for the knee joint based on a surgical plan, and the CASS 100 can depict how such angles will be affected if contemplated revisions to the surgical plan are made. As the workflow progresses to preparation of bone cuts or resections, the Display 125 can depict the planned or recommended bone cuts before any cuts are performed.

In some examples, one or more surgical planning models may be incorporated into the CASS 100 and used in the development of the surgical plans provided to the surgeon 111. The term “surgical planning model” may refer to software that simulates the biomechanics, kinematic, and/or the like performance of anatomy under various scenarios to determine the optimal way to perform cutting and other surgical activities. For example, for knee replacement surgeries, the surgical planning model can measure parameters for functional activities, such as deep knee bends, gait, etc., and select cut locations on the knee to optimize implant placement. One example of a surgical planning model is the LIFEMOD® simulation software from Smith & Nephew, Inc. In some examples, the Surgical Computer 150 includes computing architecture that allows full execution of the surgical planning model during surgery. As an alternative to full execution of the surgical planning model, in some examples, a set of transfer functions are derived that simplify the mathematical operations captured by the model into one or more predictor equations. Then, rather than execute the full simulation during surgery, the predictor equations are used. Further details on the use of transfer functions are described in U.S. patent application Ser. No. 17/269,091, entitled “Patient Specific Surgical Method and System,” the entirety of which is incorporated herein by reference in the present disclosure.

FIG. 2B shows examples of some of the types of data that can be provided to the Surgical Computer 150 from the various components of the CASS 100. FIG. 2C illustrates a “cloud-based” implementation in which the Surgical Computer 150 is connected to a Surgical Data Server 180 via a Network 175.

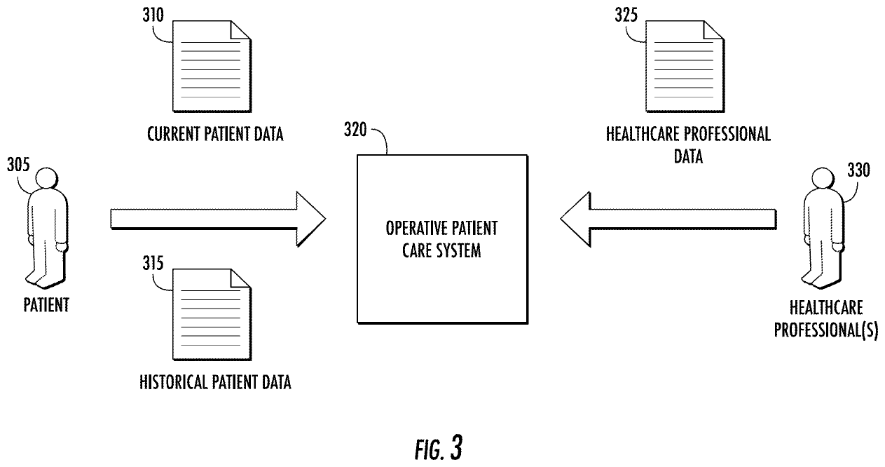

The general concepts of optimization may be extended to the entire episode of care using an Operative Patient Care System 320 that uses the surgical data, and other data from the Patient 305 and Healthcare Professionals 330 to optimize outcomes and patient satisfaction as depicted in FIG. 3.

The Operative Patient Care System 320 is designed to utilize patient specific data, surgeon data, healthcare facility data, and historical outcome data to develop an algorithm that suggests or recommends an optimal overall treatment plan for the patient's entire episode of care (preoperative, operative, and postoperative) based on a desired clinical outcome. In addition to utilizing statistical and mathematical models, simulation tools (e.g., LIFEMOD®) can be used to simulate outcomes, alignment, kinematics, etc. based on a preliminary or proposed surgical plan, and reconfigure the preliminary or proposed plan to achieve desired or optimal results according to a patient's profile or a surgeon's preferences.

Data derived from simulation of the procedure may be captured. Simulation inputs include implant size, position, and orientation. Simulation can be conducted with custom or commercially available anatomical modeling software programs (e.g., LIFEMOD®, AnyBody, or OpenSIM).

Historical data sets from the online database are used as inputs to a machine learning model such as, for example, a convolutional neural network (CNN), recurrent neural network (RNN), or other form of artificial neural network. As is generally understood in the art, an artificial neural network functions similar to a biologic neural network and includes a series of nodes and connections. The machine learning model is trained to predict one or more values based on the input data. For the sections that follow, it is assumed that the machine learning model is trained to generate predictor equations. These predictor equations may be optimized to determine the optimal size, position, and orientation of the implants to achieve the best outcome.

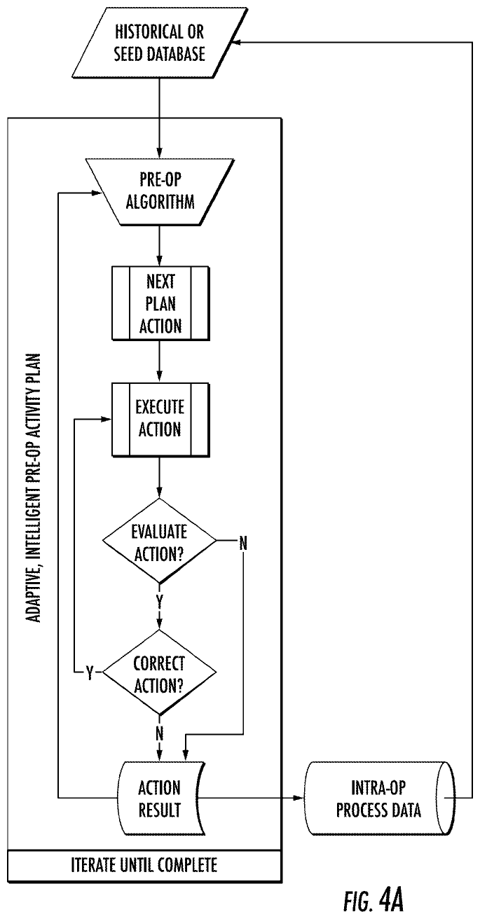

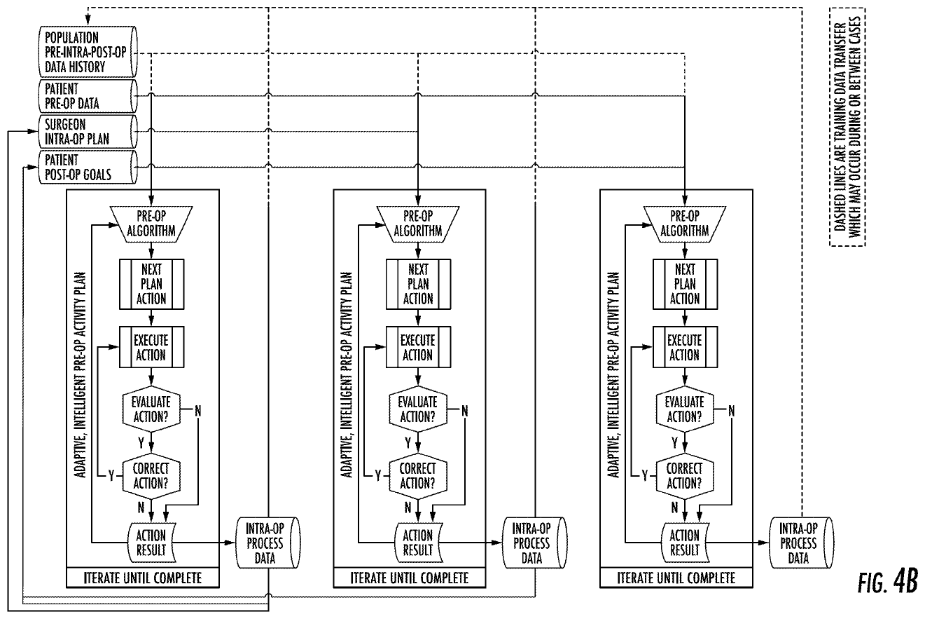

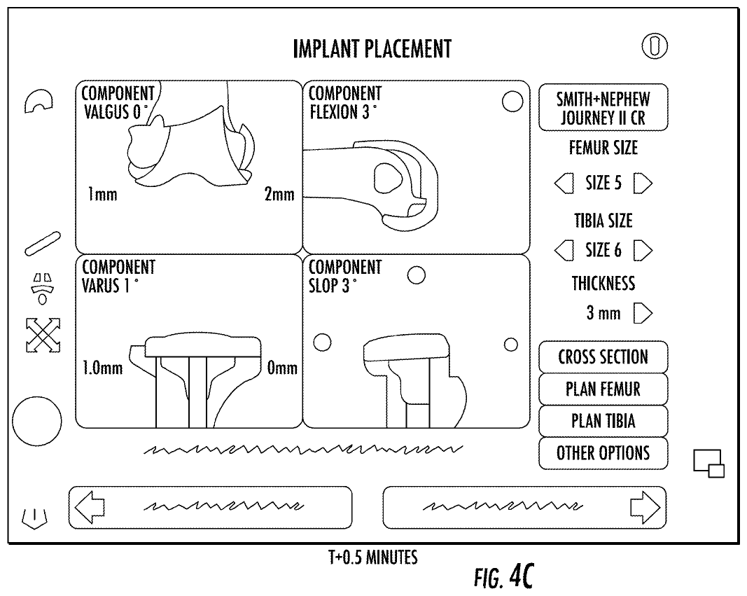

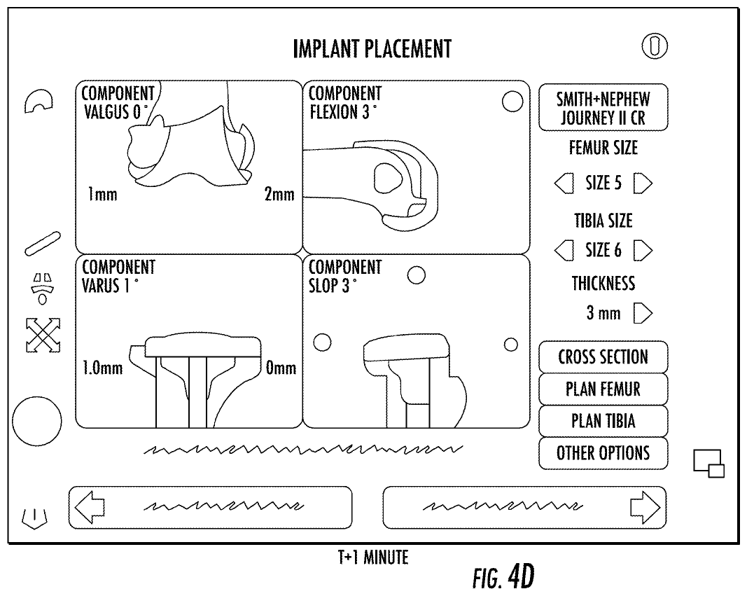

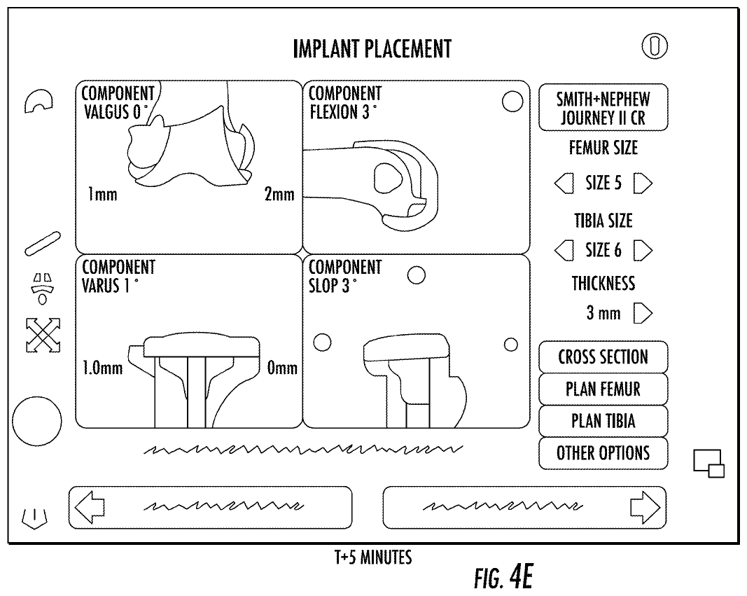

FIG. 4A illustrates how the Operative Patient Care System 320 may be adapted for performing case plan matching services. In this example, data is captured relating to the current patient 310 and is compared to all or portions of a historical database of patient data and associated outcomes 315. Once the case plan has been fully executed all data associated with the case plan, including any deviations performed from the recommended actions by the surgeon, are stored in the database of historical data. In some examples, the system utilizes preoperative, intraoperative, or postoperative modules in a piecewise fashion, as opposed to the entire continuum of care. In other words, caregivers can prescribe any permutation or combination of treatment modules including the use of a single module. These concepts are illustrated in FIG. 4B and can be applied to any type of surgery utilizing the CASS 100.

Training of the machine learning model can be performed as follows. The overall state of the CASS 100 can be sampled over a plurality of time periods for the duration of the surgery. The machine learning model can then be trained to translate a current state at a first time period to a future state at a different time period. By analyzing the entire state of the CASS 100 rather than the individual data items, any causal effects of interactions between different components of the CASS 100 can be captured. In some examples, a plurality of machine learning models may be used rather than a single model. In some examples, the machine learning model may be trained not only with the state of the CASS 100, but also with patient data (e.g., captured from an EMR) and an identification of members of the surgical staff. This allows the model to make predictions with even greater specificity. Moreover, it allows surgeons to selectively make predictions based only on their own surgical experiences if desired.

In some examples, predictions or recommendations made by the aforementioned machine learning models can be directly integrated into the surgical workflow. For example, in some examples, the Surgical Computer 150 may execute the machine learning model in the background making predictions or recommendations for upcoming actions or surgical conditions. A plurality of states can thus be predicted or recommended for each period. For example, the Surgical Computer 150 may predict or recommend the state for the next 5 minutes in 30 second increments. Using this information, the surgeon can utilize a “process display” view of the surgery that allows visualization of the future state. For example, FIGS. 4C-4E depict a series of images that may be displayed to the surgeon depicting the implant placement interface. The surgeon can cycle through these images, for example, by entering a particular time into the display 125 of the CASS 100 or instructing the system to advance or rewind the display in a specific time increment using a tactile, oral, or other instruction.

Use of a point probe is described in U.S. patent application Ser. No. 14/955,742 entitled “Systems and Methods for Planning and Performing Image Free Implant Revision Surgery,” the entirety of which is incorporated herein by reference. Briefly, an optically tracked point probe may be used to map the actual surface of the target bone that needs a new implant. This is referred to as tracing or “painting” the bone. The collected points are used to create a three-dimensional model or surface map of the bone surfaces in the computerized planning system. The created 3D model of the remaining bone is then used as the basis for planning the procedure and necessary implant sizes. An alternative technique that uses X-rays to determine a 3D model is described in U.S. Patent Application Ser. No. 16/387,151, filed Apr. 17, 2019 and entitled “Three Dimensional Selective Bone Matching,” the entirety of which is incorporated herein by reference in the present disclosure.

As noted above, in some examples, a 3D model is developed during the pre-operative stage based on 2D or 3D images of the anatomical area of interest. In such examples, registration between the 3D model and the surgical site is performed prior to the surgical procedure. The registered 3D model may be used to track and measure the patient's anatomy and surgical tools intraoperatively.

Included herein are one or more workflows representative of exemplary methodologies for performing novel features of the disclosed examples. While, for purposes of simplicity of explanation, the one or more methodologies shown herein are shown and described as a series of acts, those skilled in the art will understand and appreciate that the methodologies are not limited by the order of acts. Some acts may, in accordance therewith, occur in a different order and/or concurrently with other acts from that shown and described herein. For example, those skilled in the art will understand and appreciate that a methodology could alternatively be represented as a series of interrelated states or events, such as in a state diagram. Moreover, not all acts illustrated in a methodology may be required for a novel implementation. Blocks designated with dotted lines may be optional blocks of a workflow.

A workflow or portions (or steps) thereof may be implemented in software, firmware, hardware, or any combination thereof. In software and firmware examples, one or more workflow steps (or logic flow) may be implemented by computer executable instructions stored on a non-transitory computer readable medium or machine readable medium (for instance, executed by CASS 100 or similar system). The examples are not limited in this context.

FIG. 5 illustrates an example of a surgical workflow 500 in accordance with one or more features of the present disclosure. Workflow 500 may be representative of some or all of the operations executed by or according to any preceding or subsequent example described in the present disclosure. For example, workflow 500 may include processes for optimizing patellofemoral tracking and placement of the patellar implant through patient anatomy characterization.

At block 502, workflow 500 may include registration of patient anatomy. In any preceding or subsequent example, registration may include measuring, gathering, or otherwise determining information associated with a femur, tibia, patella, surface mapping, ROM collection, and/or the like.

For example, after femur and tibia bone tracking hardware is fixed to each bone, a registration process may be initiated. In some examples, registration may include using a point probe at several points for collection of registration information, including, without limitation, the ankle center, the hip center, landmarks on the femoral condyles, the knee center, and/or tibia landmark points. References axes, such as trans-epicondylar axis, femoral AP axis and posterior condylar axis, may be defined during registration for later use, such as during implant planning, component placement, and/or the like.

In any preceding or subsequent example, registration may include a surface mapping step for the femoral and tibial condyles, for instance, to create a virtual 3D representation of patient anatomy, generated from the collected points.

Conventional computer-assisted surgical techniques do not include patella registration or creating a virtual 3D representation of the patellar anatomy. Accordingly, in any preceding or subsequent example, the workflow 500 may include a new specific patella registration step at block 504. In any preceding or subsequent example, a device may be associated with the patella to facilitate registration. For example, a fixture device may be fixed in place on the patella, for instance, with bone pins in the patella, clamps, (temporary) adhesive, and/or the like. A non-limiting example of a patella tracking device may include devices described in International PCT Application No. PCT/US2022/051949, titled “Patella Clamp and Patella Tracking System” and filed on Dec. 6, 2022, which application claims priority to U.S. Provisional Patent Application No. 63/287,662 filed on Dec. 9, 2021, the entire contents of both applications are incorporated by reference in the present disclosure.

The fixture device may hold markers or tracking elements, such as a patella tracker array. To avoid drilling holes into the patella, markers can be placed on a patellar clamp instrument or on an instrumented patellar trial with sensors and used to track the movement of a patella throughout the range of motion. A sensor can also be imbedded into an adhesive tape or plastic sheet that is placed over the patella before the incision, to track the movement of a patella throughout the range of motion. A smart knee brace can be used before the procedure, with smart sensors on the femur and tibia built into the brace, to track the pre-operative movement of a patella. The patella registration may be associated with a patella library of previously registered patella of other patients and associated information. Examples are not limited in this context.

The patella registration step of block 504 may be configured, for instance, using the same or similar CT-free registration process as employed with the femur and tibia, using the tracking hardware on the patella. The patella can be characterized via collection of a few key patella landmarks including, without limitation, patellar medial-lateral (ML) width, superior-inferior (SI) height, AP thickness, patellar ridge, and/or the like.

Workflow 500 may include an anatomy modelling step at block 506. For example, anatomy modelling may be or may include generating 3D models of patient anatomy including, without limitation, a femur, a tibia, a patella, ligament attachments, axes, and/or portions thereof.

For instance, once the femur, tibia, and patella key landmarks and surface collections are registered, corresponding bone atlas models may be generated, a virtual 3D representation of patient anatomy may be created, and/or the like. The femur and tibia both include a substantial surface collection, which may facilitate the generation of the matching bone atlas and/or other models.

One or more patient factors may be used to build a patient risk profile. Non-limiting examples of patient factors may include age, gender, ethnicity, weight, height, body mass index (BMI), clinical characteristics, radiological findings (patellar height alta/baja, patellar tilt, patellar shift, posterior patellar angle, elongated patellar tendons, trochlear dysplasia, femoral sulcus angle, and/or the like), combinations thereof, variations thereof, and/or the like. In any preceding or subsequent example, a 3D model of the patella can be generated based upon the pre-operative characterization and/or the patient risk profile.

Virtual 3D models of the patella can be generated before or during the surgery using a non-invasive image-based approach. In any preceding or subsequent example, 3D models of the patella may be reconstructed from medical image data, such as pre-operative X-rays, CT scans, MRI, intra-operative fluoroscopy, and/or the like. Other methods may be used for 3D reconstruction of the patella such as using two or more bi-planar X-ray images, motion capture, using a collection of patella shapes with average shape geometry, ultrasound imaging, laser surface scanning, combinations thereof, variations thereof, and/or the like.

Another method to create a virtual 3D representation of the patellar anatomy and capture patellar tracking may include predicting the patellar bone size, shape, and patellar movement. A “phantom patella” may be generated by using the standard 3D collection of femur and tibia data. Various relationships, such as the relationship between bone sizes (femur and tibia size) and gender may be used to predict a range of expected patellar size. There are significant differences in mean measurements of the femur between genders. For instance, males typically have larger knees, with a larger AP dimension of the femur than females. Males typically also have wider femurs, with a wider ML dimension than females. The overall patellar width, height, and thickness of female patients are typically smaller than the overall patellar width, height, and thickness of male patients.

The 3D collection of femur data can be used to constrain the possible size and shape of the patella. For example, if the femoral sulcus angle is 145 degrees, the corresponding posterior patellar angle is predicted to be approximately 140 degrees and cannot be larger than 145 degrees. The femoral facet asymmetry, the ratio of the length of the lateral trochlear facet to the length of the medial trochlear facet, may indicate the corresponding patellar facet asymmetry. The patellar facet asymmetry and the position of the posterior ridge may be used to identify the patellar shape (Type I, II or III, equally or not-equally sized facets, and/or the like). The femoral trochlear groove depth may be used to estimate the ML width of the patella, patellar thickness, and/or other patella characteristics.

Intraoperatively, the femoral landmarks and geometry are collected for use in femur bone atlas generation. While these anatomic points are being registered, the patella can be characterized via collection of a certain patella landmarks, including, without limitation, patellar ML width, SI height, anterior-posterior (AP) thickness, posterior patellar ridge, and/or the like. These patella landmarks could be collected without the use of a patellar tracker, for instance, determined relative to the femur tracker in a static pose. Patella landmarks may provide key measurements to predict certain patellar characteristics, including, without limitation, patellar bone size, shape, patellar movement, and/or the like.

Regarding the Wiberg classification and patella types, patella type II is the most frequent patellar shape, encountered in approximately 65% of the population. An average size phantom patella type II may be generated first and then adjusted for size (e.g., ML width, SI height, AP thickness, the position of the posterior ridge, and/or the like) and patellar type (Type I—equal size of the medial and lateral patellar facets, or Type III—much smaller medial facet than lateral facet) using patient femur data and/or from the key datapoints collected.

A “phantom patella” may also be generated by referencing an anatomic database which may contain a large number of patella morphologies, different sizes, different shapes, beyond the typical three patellar types. The datapoints collected may be correlated with the dataset, and a matching statistical patella representation may be generated.

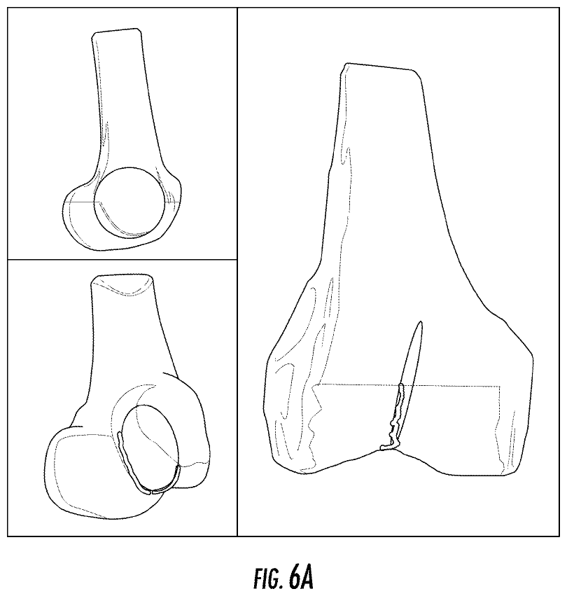

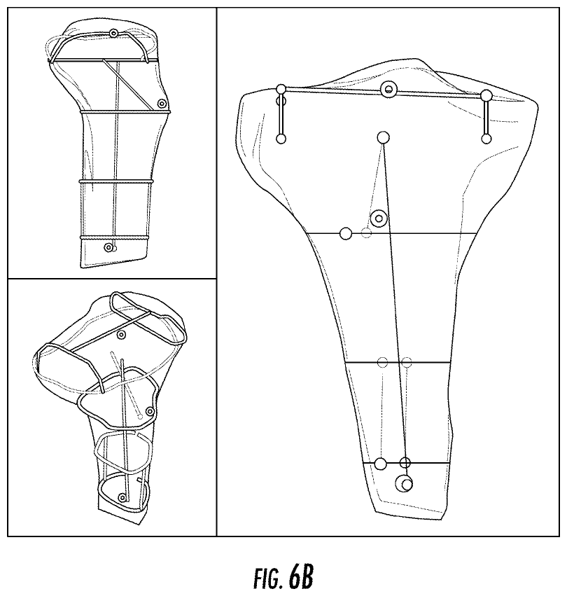

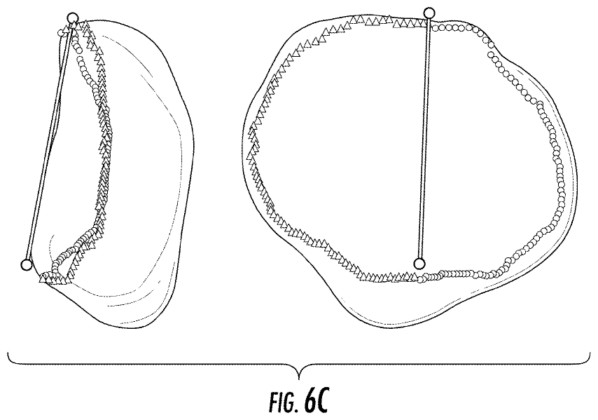

At block 508, workflow 600 may include a landmarking step. For example, from the generated 3D models, a more detailed automatic landmarking process may be applied to the solids, including many landmarks not accessible to the point probe depending on procedure. FIG. 6A depicts illustrative automatic landmarking points for a femur in accordance with one or more features of the present disclosure. Non-limiting landmarking points for the femur may include shaft centroid, epicondyles (medial+lateral), distal condyles (medial+lateral), posterior condyles (medial+lateral), anterior condyles (medial+lateral), sulcus slice lines (medial to lateral), distal sulcus point, femoral trochlear groove circle, and/or condyle articulation circles (J-Curves).

FIG. 6B depicts illustrative automatic landmarking points for a tibia in accordance with one or more features of the present disclosure. Non-limiting landmarking points for the tibia may include condyle extremity (medial+lateral), anterior plateau points (medial+lateral), posterior plateau points (medial+lateral), proximal horn, shaft centroid, tibial tubercle (center and medial third), and/or condyle articular center (medial+lateral).

FIG. 6C depicts illustrative automatic landmarking points for a patella in accordance with one or more features of the present disclosure. Non-limiting landmarking points for the patella may include, width points (medial+lateral), height points (superior+inferior), depth/thickness points (anterior+posterior), inferior articular point, articular perimeter points, posterior ridge points, centroid of the patella, patella neutral point (for instance, aligned with posterior ridge and facet midpoint line), and/or the like.

Workflow 500 may include a morphological characterization step at block 510. In any preceding or subsequent example, the morphological characterization may include characterization of the patella, femur (e.g., femoral trochlear groove), patellar tracking, and/or the like. For example, patella and femoral trochlear groove measurements may be automatically calculated using landmarks automatically determined according to any preceding or subsequent example (for instance, step 508). From the generated 3D model of the patella, a full morphological characterization can be performed by calculating several key patella measurements. The auto-landmarking process, patellar and/or femoral landmarks, and/or measurements may be the same or substantially similar to the processes described in International Patent Application Publication No. WO 2022/076773, titled “Automatic Patellar Tracking in Total Knee Arthroplasty” and filed on Oct. 8, 2021, the entire contents of which are incorporated by reference in the present disclosure.

Various patella measurements may be determined according to any preceding or subsequent example. Non-limiting examples of patella measurements may include one or more of the following: Patella Width (mm) measured between the most lateral point of the articulating surface and the most medial point of the articulating surface; Maximum Patella Height (mm) measured between the most inferior point and the most superior point (base), along the z-axis; Patella Thickness (mm), measured between the most projecting point on the anterior surface and the most posterior point on the vertical ridge; Lateral Facet Width (mm), measured between the center point and the most lateral point of the articulating surface; Medial Facet Width (mm), measured between the center point and the most medial point of the articulating surface; Height of the Articular Surface (mm), measured between the most superior point (base) and the point on the margin of the inferior articular surface; Quadriceps Angle, or Q-angle, (degrees) is calculated using the femoral, tibial and patella landmarks. The angle is calculated in the frontal plane at the intersection of 2 lines: one line from the femoral proximal landmark to the center of the patella and a second line from the center of the patella to the tibial tubercle landmark; and/or The Posterior Patellar Angle, the angle between the medial and lateral normals.

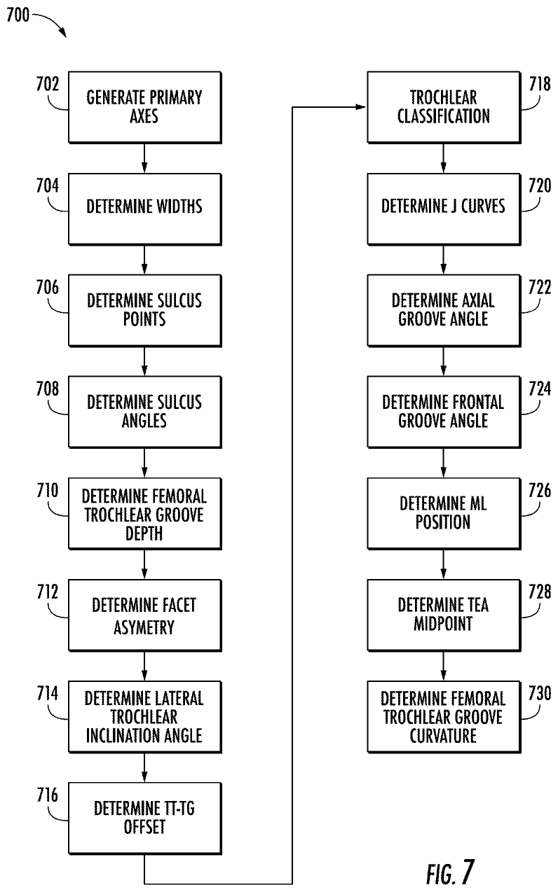

In any preceding or subsequent example, the femoral trochlear groove may be characterized. FIG. 7 illustrates an example of a workflow 700 for characterizing a femoral trochlear groove and associated patient anatomy. The workflow 700 may be used for characterizing a trochlear groove of both native patient anatomy and an implant device (for instance, the post-operative state).

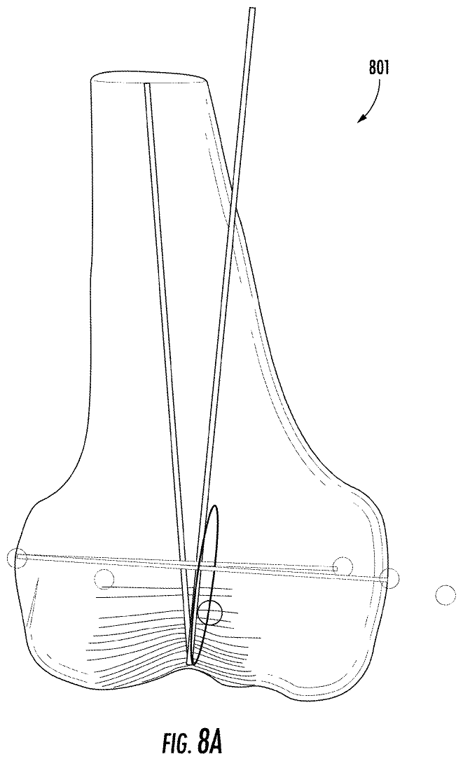

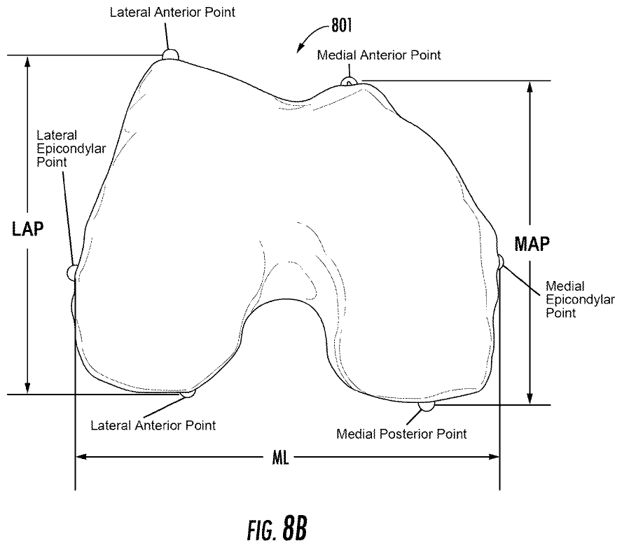

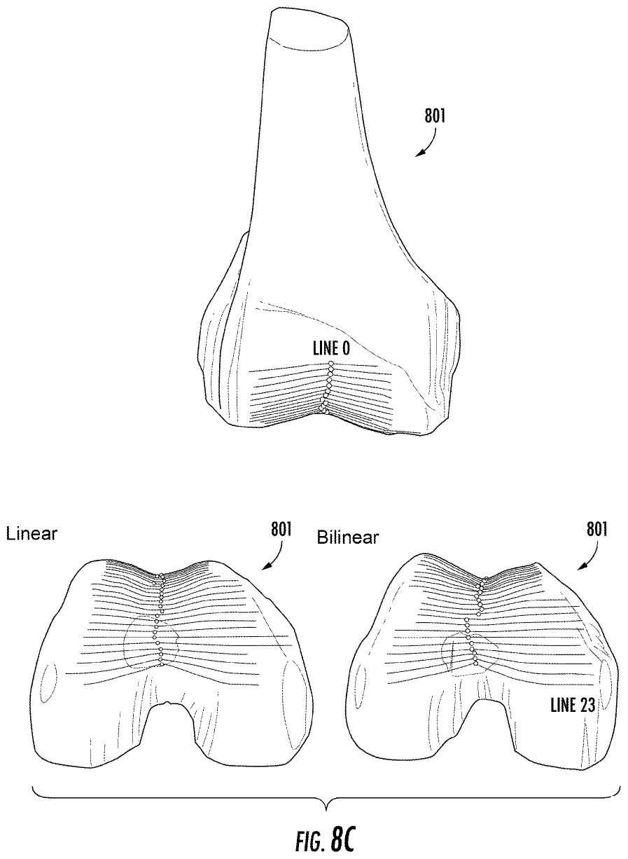

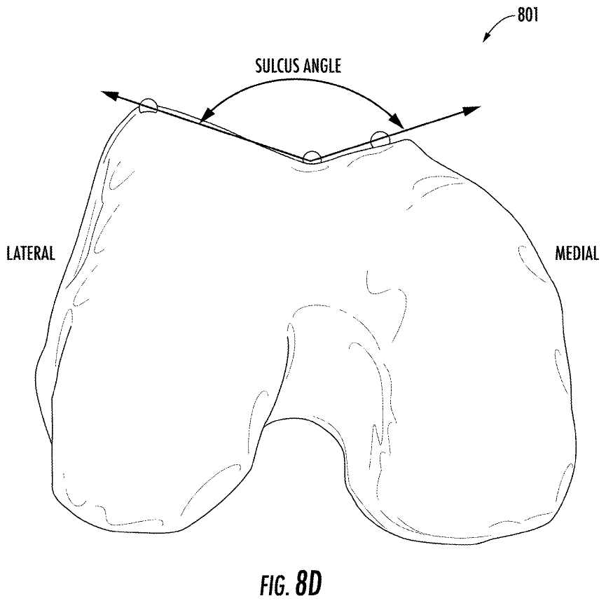

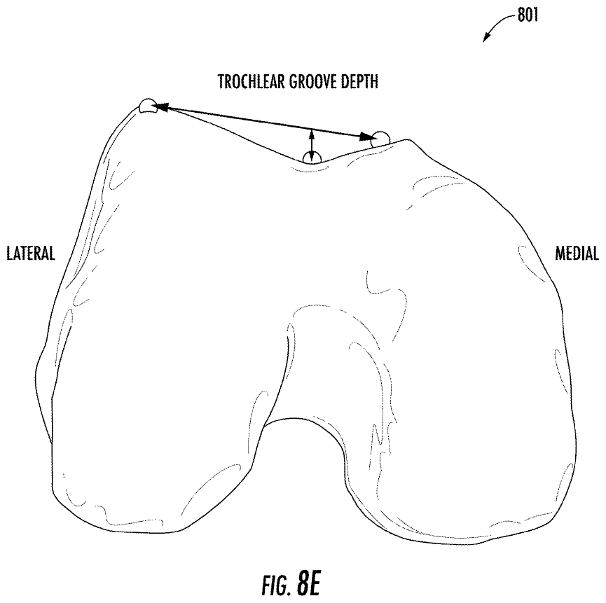

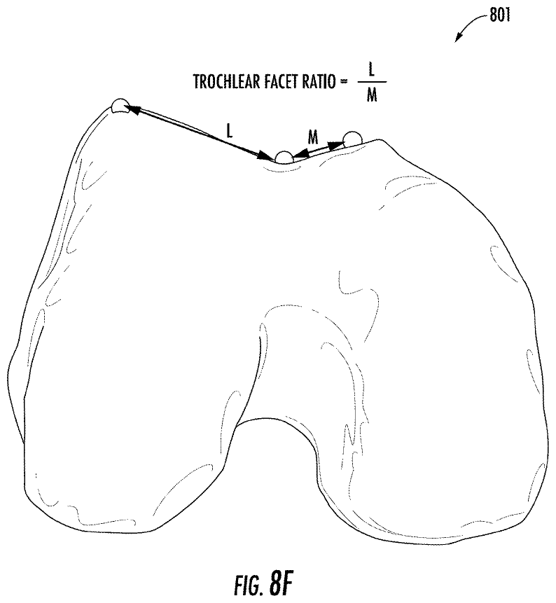

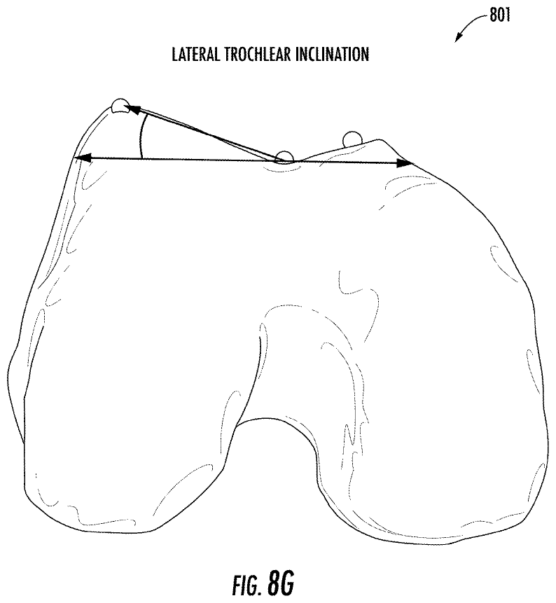

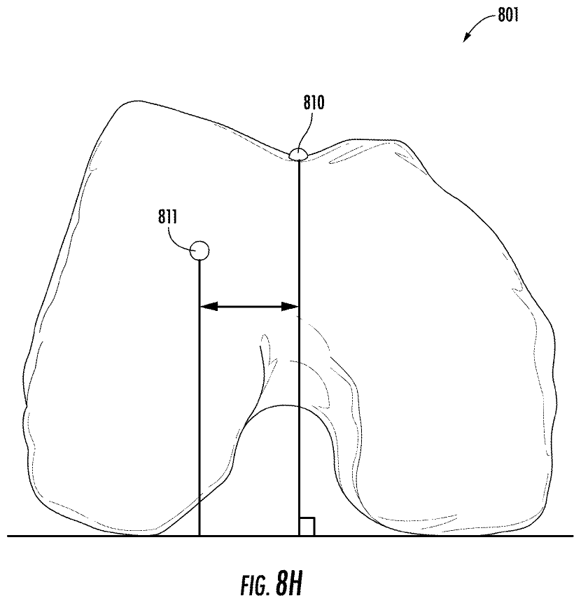

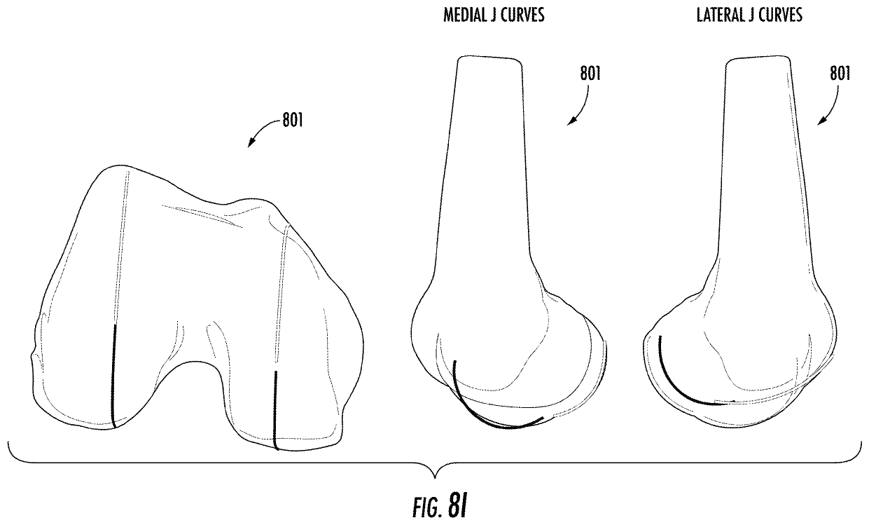

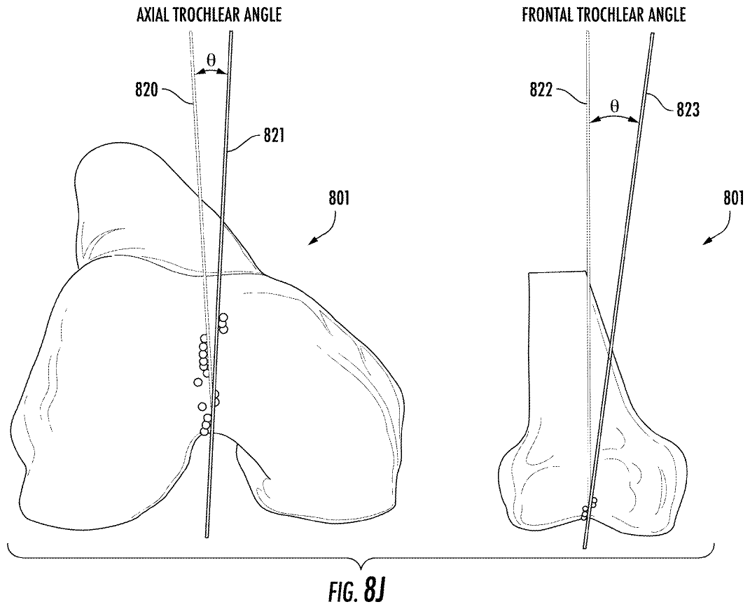

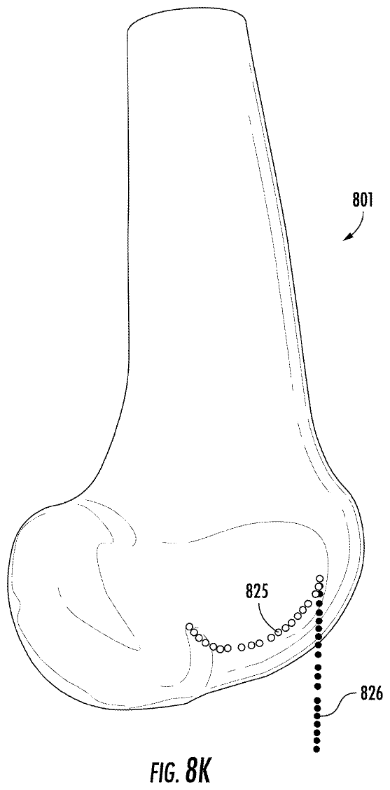

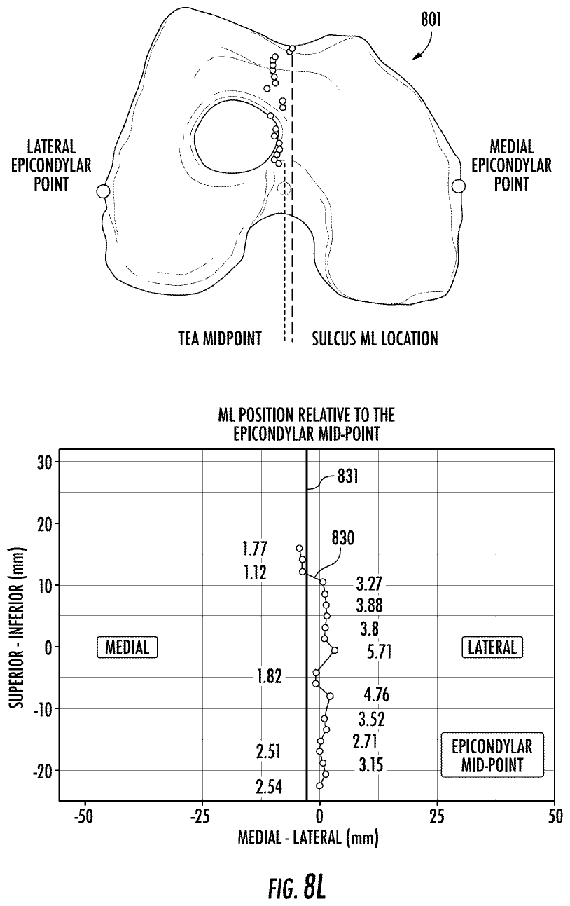

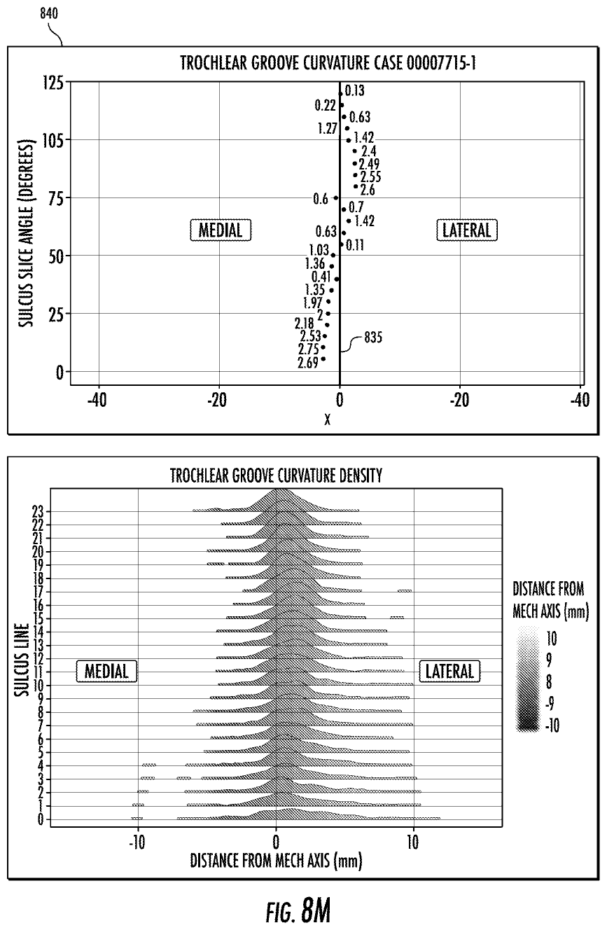

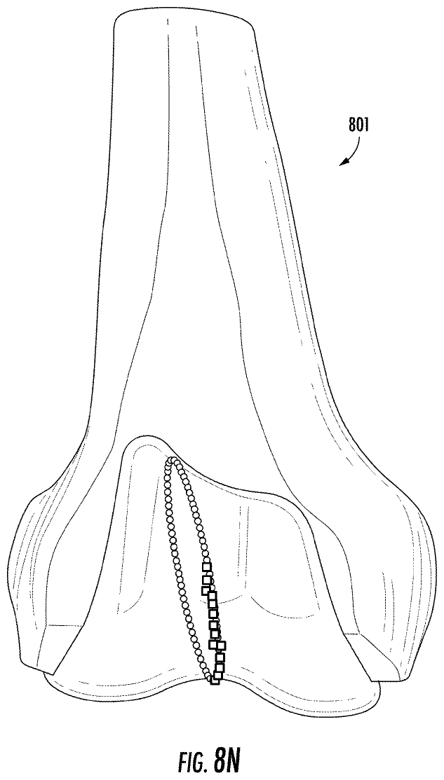

As shown in FIG. 7, workflow 700 may include generating primary axes at block 702. For example, a femur bone model may be generated during the point registration step 502 of the workflow 500. The primary axes of the patient may be generated from the point registration landmarks collected intraoperatively. FIG. 8A depicts an illustrative femur model showing axes for a femur in accordance with one or more features of the present disclosure. As shown in FIG. 8A, a femoral bone model 801 may be generated from the bone registration step 502 and positioned relative to the axes generated intraoperatively. Non-limiting axes for the femur may include intramedullary axis, trans epicondylar axis, posterior condylar axis, mechanical axis, AP axis, and/or femur sulcus angles (per sulcus slice). Non-limiting axes for the tibia may include intramedullary axis, mechanical axis, tibial plateau, plateau axes (anterior, posterior, and center), condylar profiles (medial+lateral), tubercle plane and axis, and/or anterior and posterior cortex axes.

At block 704, the workflow 700 may include determining widths of the femur. FIG. 8B depicts points for determining femoral size of a femur model in accordance with the present disclosure. In reference to the model depicted in FIG. 7B, AP width of the lateral and medial condyles may be calculated as the distance between the most posterior point and most anterior point on each condyle. The ML width may be calculated as the distance between the medial epicondylar and lateral epicondylar point. In any preceding or subsequent example, the femoral size may be defined by the ML and the anterior-posterior widths for each condyle (LAP and MAP).

Workflow 700 may include determining sulcus points at block 706. FIG. 8C depicts sulcus points and sulcus lines for a femur model in accordance with the present disclosure. As shown in FIG. 8C, sulcus points, the deepest points in the femoral trochlear groove, and sulcus lines, contour lines captured on the surface of the femoral trochlear groove, may be captured by sweeping along the femoral trochlear groove surface of femur model 801.

Workflow 700 may include determining sulcus angles at block 708. FIG. 8D depicts determining a sulcus angle for a femur model in accordance with the present disclosure. In any preceding or subsequent example, a sulcus angle may be calculated for all the sulcus lines. The sulcus angle is defined as the angle between the most prominent condylar point on the medial aspect of the sulcus line and the lateral aspect of the sulcus line.

Workflow 700 may include determining a femoral trochlear groove depth at block 710. FIG. 8E depicts determining a femoral trochlear groove depth for a femur model in accordance with the present disclosure. For instance, for each sulcus line, the femoral trochlear groove depth may be calculated as the distance between the line connecting the medial and lateral prominence points and the deepest sulcus point.

Workflow 700 may include determining facet asymmetry at block 712. FIG. 8F depicts determining facet asymmetry for a femur model in accordance with the present disclosure. For instance, for each sulcus line, the facet asymmetry may be calculated as the ratio of the length of the lateral trochlear facet to the length of the medial trochlear facet.

Workflow 700 may include determining a lateral trochlear inclination angle at block 714. FIG. 8G depicts determining a lateral trochlear inclination angle for a femur model in accordance with the present disclosure. For instance, for each sulcus line, the lateral trochlear inclination angle is calculated as the angle between a line fit to the lateral trochlear facet and a horizontal line passing through the sulcus point in the axial plane.

Workflow 700 may include determining a Tibial Tubercle-Trochlear Groove (TT-TG) offset at block 716. FIG. 8H depicts determining a TT-TG offset for a femur model in accordance with the present disclosure. For instance, the TT-TG offset offset/distance may be the distance between the following: parallel lines in the axial plane: (a) perpendicular from the posterior condylar reference line to the deepest point of the trochlea and (b) perpendicular from the posterior condylar reference line to the apex of the tibial tubercle. Referring to FIG. 8H, the TT-TG offset may be the distance between the deepest sulcus point 810 and the tibia-tubercle 811 in the axial plane.

At block 718, workflow 700 may include femoral trochlear groove classification. In any preceding or subsequent example, the trochlear classification may be or may include determining a geometric abnormality of the femoral trochlear groove that can result in abnormal patellar tracking along the trochlea. The geometric abnormality may be or may be determined the same or similar to techniques described in Elias D.A., “Patellofemoral Joint,” Measurements in Musculoskeletal Radiology, Medical Radiology (2020). The values for the following measurements for the first sulcus line may be used for determining whether a case is abnormal in regard to trochlear dysplasia: sulcus angle>145°, trochlear depth<3 mm, lateral inclination angle<11°, trochlear facet asymmetry>1.7. In any preceding or subsequent example, if one or more of the values of the sulcus angle, trochlear depth, lateral inclination angle, or the trochlear facet asymmetry disagree, only the sulcus angle and depth may be used to identify abnormal cases.

At block 720, workflow 700 may include determining J-Curves. FIG. 8I depicts determining J-Curves for a femur model in accordance with the present disclosure. In any preceding or subsequent example, the J-Curves may be calculated by fitting arcs to the end points of the sulcus lines, the most prominent points along the condylar surface. The femoral J-Curves may be constrained to the neutral femoral plane and multiple arcs can be captured for each condyle; the radius of each J-Curve may be extracted.

At block 722, workflow 700 may include determining an axial groove angle. At block 724, the workflow 700 may include determining a frontal groove angle. FIG. 8J depicts determining an axial groove angle and a frontal groove angle for a femur model in accordance with the present disclosure. For instance, the axial groove angle may be calculated as the angle between the femoral AP axis and trochlear fitted plane fit to the sulcus points, for instance, generated at block 706, in the axial plane. Referring to FIG. 8J, the axial trochlear angle is measured as the angle, Φ, between the AP axis (820) and the plane fitted to the sulcus points (821) in the axial plane; the frontal trochlear angle is measured as the angle, θ, between the femoral mechanical axis (822) and the plane fitted to the sulcus points (823) in the frontal plane.

At block 726, workflow 700 may include determining an ML position of the femoral trochlear groove. FIG. 8K depicts determining an ML position of the femoral trochlear groove for a femur model in accordance with the present disclosure. For instance, the ML position of the femoral trochlear groove may be calculated by determining the distance between the sulcus points and reference axes along the ML axis. Referring to FIG. 8K, sulcus points 825 may be projected onto the same plane 826. Prior to calculating the ML position, the sulcus points may be unrolled and projected into the same plane. Sulcus point projection may allow for the position of sulcus points relative to one another to be viewed in one plane. Such a visualization may, inter alia, aid surgeons in understanding the sulcus position throughout the range of motion.

At block 728, workflow 700 may include determining a trans-epicondylar axis midpoint (TEA Midpoint). FIG. 8L depicts determining a TEA midpoint for a femur model in accordance with the present disclosure. The TEA midpoint may be defined as the midpoint between the medial and lateral epicondylar points. The distance between the unrolled sulcus points and the epicondylar midpoint may be calculated and plotted. Similarly, the ML distance between the femoral mechanical axis and the sulcus points may be calculated. Referring to FIG. 8L, therein is depicted the epicondylar midpoint and a plot of the unrolled sulcus points (830) and their ML distance from the epicondylar midpoint (831).

At block 730, workflow 700 may include determining a femoral trochlear groove curvature. FIG. 8M depicts determining a femoral trochlear groove curvature for a femur model in accordance with the present disclosure. More specifically, FIG. 8M depicts plot 840 of a femoral trochlear groove curvature for a single bi-linear femur case and plot 841 of an averaged femoral trochlear groove curvature for multiple femur cases.

The femoral trochlear groove curvature is a two-dimensional plot showing the curvature of the deepest sulcus points and the distance on the x-axis from a reference line (the mechanical axis) (line 835 of plot 840). In order to visualize the linear or bi-linear behavior of the femoral trochlear groove, the sulcus points may be plotted for each sulcus line for each case. Referring to plot 841 of FIG. 8M, as the femur goes from full extension (line 1) to full flexion (line 24), the position of the deepest sulcus point shifts from medial to lateral relative to the femoral mechanical axis. Overall, the positions of the deepest sulcus points are lateral to the femoral mechanical axis as indicated by plots 840 and 841 of FIG. 8M.

Referring to FIG. 5, the morphological characterization 510 of workflow 500 may include an implant trochlear groove characterization process. In any preceding or subsequent example, determining the implant trochlear groove characteristics may include generating an implanted sulcus and determining the measurements, calculations, and/or the like generated via the steps of the workflow 700 for the femoral implant (for instance, for characterizing a native femur, the model 801 may be a model of the patient anatomy; for characterizing a femur implant component, the model 801 may be a model associated with the femur implant component). After determining the femoral trochlear groove measurements via workflow 700, the implant trochlear groove characterization process may include determining the ML shift between the native and implant sulcus.

FIG. 8N depicts characterizing a trochlear groove for an implanted femur model in accordance with the present disclosure. In any preceding or subsequent example, the axial groove angle, the frontal groove angle, the center of the trochlear circle and radius, J-curves radii, sulcus location (ML distance from the epicondylar midpoint or the femoral mechanical axis) may be collected before the surgical procedure (native) and after the procedure (implanted). The collected information for both the implanted and native femoral sulcus can be used to optimize the implant position and orientation, which may provide, among other things, better visualization, for instance, to help surgeons in understanding the sulcus position and orientation before and after surgical procedure, and different surgical scenarios/options that may be available.

Referring to FIG. 5, the workflow 500 may include risk classification at block 512. In any preceding or subsequent example, patient information may be used to determine risk information for a patient. In any preceding or subsequent example, the risk information may be or may include a risk classification and/or risk profile associated with potential outcomes of a knee arthroplasty procedure. In any preceding or subsequent example, the risk information may be used to predict an outcome of a knee arthroplasty procedure (or a portion or characteristic thereof) for a patient.

In any preceding or subsequent example, the patient information may include, without limitation, demographic information, clinical characteristics, medical diagnostic findings (for instance, medical imaging results), morphological characterization, and/or the like. The patient information may be used to generate a patient risk profile according to any preceding or subsequent example. For example, three risk profiles (for instance, in one non-limiting example, High, Medium, and Low Risk) can be generated based on patient factors, including, without limitation, radiological evaluation, morphological characterization, and/or the like. Patient factors such as patient age, gender and ethnicity may also be included as elements of a risk profile. In one non-limiting example, female patients are associated with an increased risk of patella-femoral pain, therefore female patients may be classified in High and Medium risk profiles. Research indicates that female patients have 2-3 times higher incidence of patella-femoral pain compared with males due to an increased Q-angle, frontal plane alignment, lower extremity muscle strength, as well as other factors. Other patient characteristics, such as age, ethnicity, and increased body mass index have also been linked to increased patella-femoral complications. Another item of patient information may include ethnicity. For example, on average, African Americans patients may exhibit increased anteroposterior height of the femur as compared to Caucasians and East Asians. Asian patients may exhibit a greater degree of curvature of the femoral condyles. However, Implants are generally designed based on average Caucasian anatomy, which may explain an increase in patellofemoral complications, including anterior knee pain, following TKA in patients of different ethnicities.

Clinical and radiological evaluation can provide patient information that may be relevant for the assessment of patellar tracking and history of patellar dislocation. For instance, patients that have a pre-operative patellar shift of >3.0 mm have a higher likelihood of maltracking and can be classified as a High risk profile. In another example, patients with a deeper femoral sulcus groove than average have a higher risk of complications, for instance, because such patients may be more prone to patella-femoral pain.

Large patellar width and large lateral patellar width may also be risk factors. For instance, a higher Wiberg index may indicate hypoplasia in the medial patellar facet (flat) and hyperplasia in the lateral patellar facet, and, consequently, a High patient risk. In another instance, a very small patellar width and a lengthened lateral facet may indicate trochlear dysplasia or decreased femoral trochlear groove depth, which could be another factor for patello-femoral pain and patellar maltracking. In a further instance, trochlear dysplasia is a well-described risk factor for patellofemoral instability that has been found in up to 85% of patients with patellar instability and knee pain. Type I or Type II patella are common and not usually associated with a higher incidence of anterior pain, therefore patients with a Type I or Type II patella may be designated as a Low or Medium risk profile. In an additional instance, having a Type III patella and a larger patellar tilt angle are usually associated with higher incidence of anterior pain and, therefore, a higher risk.

In any preceding or subsequent example, different combinations of patient information, including, without limitation, patellar sizes and shapes, Type I, II or III, with different femoral sizes and shapes, with linear or bilinear curvatures, tibial tubercle position, Q-angle, pelvis width, soft tissue properties, and/or the like may lead to a different risk profile. For example, a patient with a Type III patella and a larger than average Q-angle maybe considered a High risk profile, for instance, predisposed to patellar maltracking and a higher incidence of anterior pain after TKA.

In any preceding or subsequent example, a risk level, score, classification, group, indicator, and/or the like may be determined and assigned to each patient, based on the calculated patient risk profile. Different risk levels or groups may be developed, depending, for instance, on the case complexity, implant characteristics, patient characteristics, and/or the like. Risk stratification may allow better surgical decision-making, set realistic patient expectations, make better use of resources, identification of high-risk groups for complications, anticipate complications, predict a clinical outcome, and help the surgeon to proactively plan the most appropriate treatment for that patient. Proactively identifying a potential high-risk group or level for a certain patient may give a direction to the surgeon in terms of implant choices, what implant would be best for that patient, modifying the level of constraint, necessary implants and instruments, surgical approaches, implant positioning, and/or the like.

In one non-limiting example, for a High risk female patient with Type III patella (high risk of dislocation), larger than average Q-angle, and a pre-operative patellar shift>3.0 depicted on X-rays, a pre-operative plan can be generated and provided to the surgeon as guidance to achieve the most optimal alignment, prevent dislocation (for instance, a deep trochlear groove, etc.) and provide maximum joint stability for that patient. Femoral resections, tibial resections, and/or optimal patella preparation (for instance, optimal patellar resection, thickness, etc.) can be provided to the surgeon. Implants size, shape, position, and/or orientation can be provided before or during the surgery to achieve the most optimal patellar tracking, ligament balance, and/or overall leg alignment for that patient.

At block 514, the workflow 500 may include a planning optimizer process. Based on the patient geometric or anatomical data collected (for instance, bony anatomy landmarks, alignment, and/or the like) and collected intraoperative data (for instance, passive flexion kinematics, gap data from a soft tissue tensioner), an implant planning optimizer may be operative to determine one or more optimal implants and/or the optimal positioning of the implants.

In any preceding or subsequent example, for instance, the selection and/or configuration of knee implant components may be determined to address patient-specific risk factors in order to directly address potential post-operative issues that are personal to a patient's unique anatomy and risk factors.

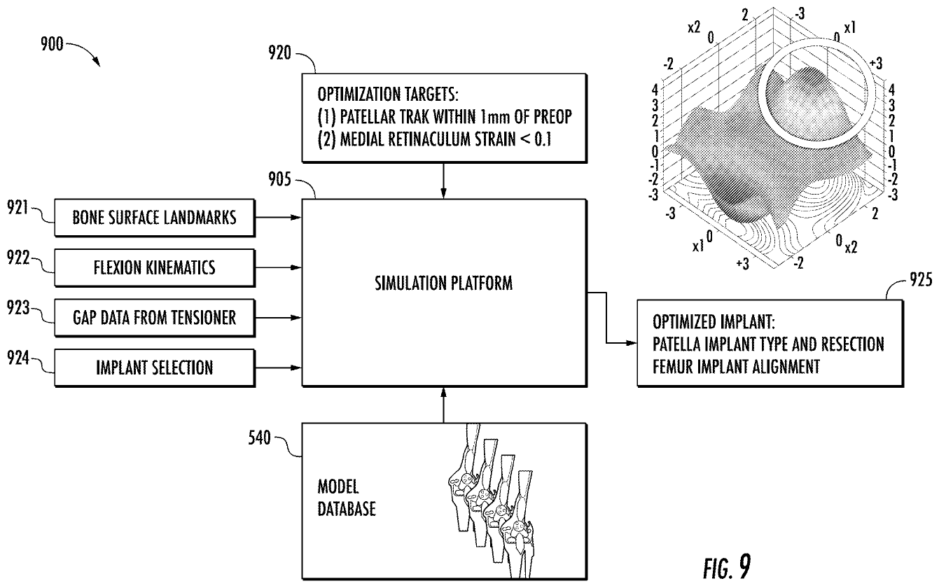

FIG. 9 depicts an illustrative operating environment for a planning optimizer process in accordance with the present disclosure. As shown in FIG. 9, an operating environment 900 may include a simulation platform 905. In any preceding or subsequent example, the simulation platform 905 may include, may be, or may be the same as the LIFEMOD® simulation software, for example, LIFEMOD® simulation software transfer functions. For example, in any preceding or subsequent example, a set of transfer functions are derived that simplify the mathematical operations captured by the models into one or more predictor equations. These transfer functions may be generated by performing a wide range of simulations of knee biomechanical performance while varying the model input parameters which define demographic (for instance, size, height, weight, etc.), clinical (for instance, strength, ROM, etc.), medical diagnostic (for instance, wear, damage, deformity, etc.), or morphological (bone geometry, angles, anatomic dimensions, etc.) characterizations, and/or the like. For each simulation model, the resulting model responses are captured to represent post-operative kinematics, rotation, laxity, strain, and alignment, and the transfer equations may be generated to capture these relationships. Then, rather than execute the full simulation during surgery, the predictor equations are used. Further details on the use of transfer functions are described in WIPO Publication No. 2020/037308, filed Aug. 19, 2019, entitled “Patient Specific Surgical Method and System,” the entirety of which is incorporated by reference into the present disclosure.

The simulation platform 905 may receive various inputs, including, without limitation, bone surface landmarks 921, flexion kinetics 922, gap data (from tensioner) 932, and/or implant selection 924. In any preceding or subsequent example, the simulation platform 905 may retrieve information from a model database 540. In any preceding or subsequent example, the simulation platform 905 may receive optimization targets 920, including, without limitation a patellar tracking target (for instance, within 1 mm pre-operative), a medial retinaculum strain (for instance, <0.1), and/or the like. The simulation platform 905 may be configured to determine an optimized implant 925 based on the input 921-924, model database 540, and optimization targets 920. In any preceding or subsequent example, the optimized implant 925 may specify various implant characteristics, for instance, a patella implant type and resection information, a femur implant alignment, and/or the like.

In any preceding or subsequent example, the model database may include simulation data for pre-run knee simulations (for instance, hundreds or thousands of simulations), for each risk group. Simulations may include patient profiles (for instance, age, gender, ethnicity, BMI, and/or the like) and activities such as standing, sitting, walking, running, walking up and down stairs, twisting, and performing deep knee bends. Within the model database 540, the models may contain or be associated with a range of varying bone landmarks, leg alignment, soft tissue properties, and/or implant selections and components placement to cover the range of patient variables. Simulation results may include post-operative patellar kinematics, post-operative soft tissue strain, joint forces, and/or the like.

In any preceding or subsequent example, for each patient profile, a response surface analysis may be performed which generates statistical equations which relate the patient factors to the model results. Using these transfer function equations, the solution for implant selection or placement may be optimized according to case specific targets.

In any preceding or subsequent example, the results of the transfer function optimization may provide an optimized range of solutions in which the optimization targets may be achieved. A user can change the implant selection parameters (for instance, size down, size up, and/or the like) and re-run the optimization process via the simulation platform to adjust or fine tune the results. In addition to the optimized implant data, the optimization process may also provide display gauges or animations of the statistically optimized simulation behavior, for example, to provide visualization of the process and/or results.

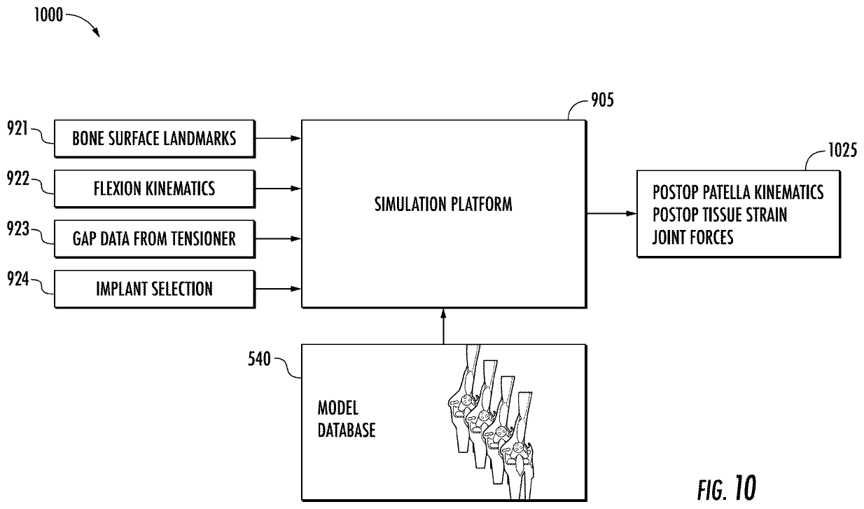

FIG. 10 depicts an illustrative operating environment for a planning optimizer process in accordance with the present disclosure. In any preceding or subsequent example, simulation platform 905 of operating environment 1000 may include, may be, or may be the same as the LIFEMOD® simulation software, for example, LIFEMOD® simulation software simulation database and lookup algorithms. Simulation platform 905 may receive one or more of inputs 921-924 and, using information from model database 540, generate output 1025 in the form of postoperative information, such as postoperative patella kinematics, postoperative tissue strain, joint forces, and/or the like.

Operating environment 1000 may facilitate the use of the model database 540 and direct lookup of pre-simulated cases via the simulation platform 905 for identification of the “best fit” case. After patellar characterization, the algorithm can choose the most closely related case in the model database 540 and predict 1025 the post-operative patellar tracking or patellar maltracking risk based on the patient's anatomy and demographics by identifying the “best fit” case in the database.

In any preceding or subsequent example, optimization may include registering the patient anatomy, performing ROM, and calculating the optimal placement for all the of the implants in order to optimize ROM and ultimately the tracking of the patellar component. Characterizing the pre-operative anatomy and range of motion, the optimal placement of the implants may be predicted based on the implant size offerings, designs, dimensions and the known correlations between anatomy and ROM.

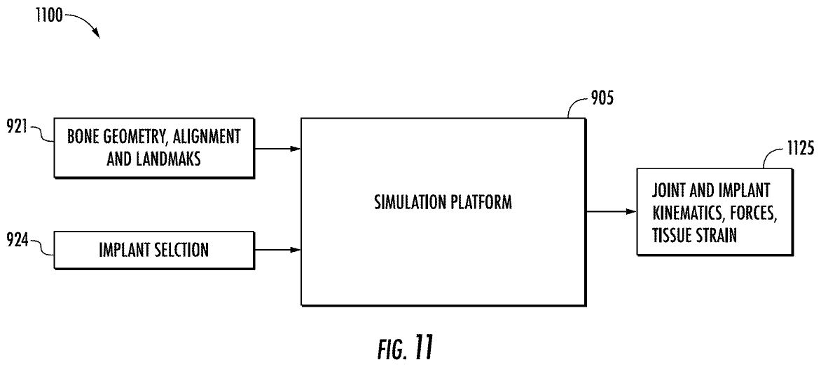

FIG. 11 depicts an illustrative operating environment for a planning optimizer process in accordance with the present disclosure. In any preceding or subsequent example, simulation platform 905 of operating environment 1100 may include, may be, or may be the same as the LIFEMOD® simulation software. In any preceding or subsequent example, operating environment 1100 may facilitate a process of patellar characterization and morphological determination within a surgical process in which 3D geometry data is already known before operation begins. The 3D geometry could be measured directly from the available patient scan data, or it could be determined by a machine learning rubric based on digitization of only a few key patellar points. The surgical process may be the same or similar to a Visionaire process provided by Smith & Nephew, Inc.

In any preceding or subsequent example, the model database 540 may be accessed via direct lookup of matching case or via transfer function optimization. In addition, or in the alternative, the patient case could be directly simulated by: loading the patient specific anatomy into the simulation platform 905; loading patient-specific ligaments onto the bones of the patient anatomy; and simulating desired knee behavior to generate output 1125, for instance, including joint and implant information, kinematics information, forces information, tissue strain information, and/or the like.

Referring to FIG. 5, once the implant information (for instance, implant selection, implant positions, implant alignment, and/or the like) has been determined via blocks 502-514 of workflow 500, the surgical steps may be performed to install the implant device(s). At block 516, the workflow 500 may include bone cutting. For example, femur and tibia preparation can be achieved by using a navigated burr in a navigated or computer-assisted surgical workflow (for instance, a CASS surgical workflow). Based on the output in the previous step, the surgeon can use conventional resection instruments to prepare the bone and place the patellar component. An alternative to patellar preparation may involve the use of a navigated burr similar to that of the femur and tibia preparation. The CASS may guide the resection depth and angle according to the implant position optimized at block 514.

At block 518, the workflow 500 may include a trial/ROM step. For example, once the bones are resected, the surgeon can trial the components and confirm their placement by conducting a range of motion assessment. At block 520, the workflow 500 may include a cement and close step. For example, once the implant selection and positions are confirmed, the surgeon can cement in the final components and close the incision to complete the surgical process.

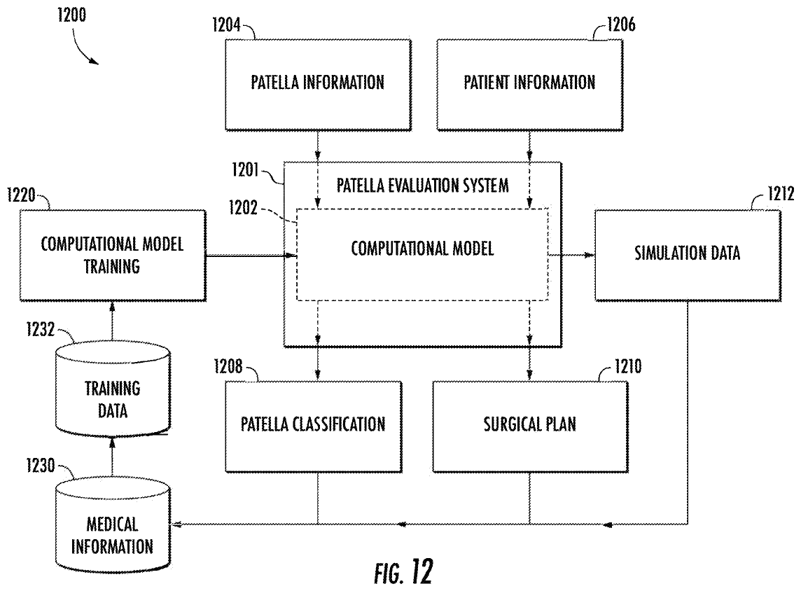

FIG. 12 depicts an illustrative operating environment for a surgical planning process in accordance with the present disclosure. As shown in FIG. 12, an operating environment 1200 may include a patella evaluation system 1201. Evaluation system 1201 may be or may include a computing device and associated processing circuitry, memory, and/or the like, for instance, the same or similar to surgical computer 150. In various examples, evaluation system 1201 may be a computer device for performing a patella evaluation process according to the present disclosure.

Evaluation system 1201 may store or access a computational model 1202. In some examples, computational model 1202 may be or may include various types of computational models including, without limitation, regression models, lookup tables, transfer functions, simulation models, virtual anatomical models, artificial intelligence (AI) and machine learning (ML) (AI/ML) models, neural networks (NN), convoluted neural network (CNN), recurrent neural network (RNN), combinations thereof, variations thereof, and/or the like.

In various examples, computational model 1202 may be configured to receive patient-specific input (e.g., patella information 1204 and/or patient information 1206) associated with evaluating a patient's patella and generating output in the form of a patella classification 1208 in the form of a risk classification for patella or patella-related complications for resulting from an arthroplasty procedure. In some examples, computational model 1202 may be configured to output a surgical plan 1210 based, at least in part, on the patella classification 1208. For example, surgical plan 1210 may include, without limitation, parameters, surgical processes, implant component information (sizes, positioning, and/or the like), and/or recommended instructions for performing surgical preparation procedures (e.g., location and/or amount of resurfacing and/or reshaping, cutting information, angles, dimensions, tools, surgical procedures, and/or the like). The surgical plan 1210 may be determined to reduce or even eliminate potential post-operative issues that are personal to a patient's unique anatomy and risk factors.

In various examples, patella information 1204 may be input into the patella evaluation system 1201 and provided to computational model 1202. In general, patella information 1204 may include any information associated with the patella of the patient that is the subject of the patella evaluation process. Patella information 1204 may include patella anatomical or physical characteristics, such as dimensions, facets, and/or the like and functional characteristics, such as location, movement, etc. during knee extension/flexion, tension, pressure, patient pain, kinematic information, performance information, and/or the like. In various examples, patella information 1204 may include information associated with other knee structures, such as the femur, tibia, muscles, tendons, femoral implant components, tibial implant components, trochlear groove information, and/or the like. In general, patella information 1204 may include any information relevant to evaluating a patella according to some examples.

In various examples, patient information 1204 may be input into the patella evaluation system 1201 and provided to computational model 1202. In general, patient information 1204 may include any information associated with the subject patient that may be relevant to evaluating the patient's patella, surgical outcomes, and/or the like. Non-limiting examples of patient information 1204 may include age, gender, physical characteristics (e.g., height, weight, etc.), medical history, knee condition (e.g., patient medical evaluation), injuries, surgeries, and/or the like.

Computational model 1202 may be trained via computational model training 1220 to receive patella information 1204 and patient information 1206 of a patient and generate a patella classification 1208. In general, patella classification 1208 includes patella risk classification that is or includes a prediction, estimation, recommendation, and/or the like associated with surgical risks based on the patella and other physical characteristics of the patient (for instance, the trochlear groove information).

Accordingly, the output of evaluation system 1201, particularly using a trained computational model 1202, may provide a practical application and real-world results for patients. In addition, evaluation system 1201 provides an improvement in the field of knee arthroplasty, and in particular, computer-based surgical systems through the ability of evaluation system 1201 to provide patella classifications 1208 in a manner that is not possible using conventional computing systems. Existing computer-based surgical systems, simulation systems (including systems that simulate the patella), and/or the like are not capable of providing a patella classification 1208 according to some examples.

Computational model training 1220 may be or may include a computer-based process for training computational model 1202 using training data 1232. Training data 1232 may include labelled and/or unlabeled data. Training data 1232 may receive and/or transform medical information 1230 into training data for training computational model 1202. Medical information 1230 may include computer-generated data (e.g., predictions, recommendations, classifications, etc.) such as patella classifications 1208, surgical plans 1210, simulation data 1212 (for instance, from simulation platform 705) modified by real-world outcomes based on real patients and/or labeled by medical professionals.

For example, training data 1232 may be generated pairing patella classifications 1208 with corresponding real-world patient outcomes (for instance, in a medical database of medical information 1230) indicating the accuracy of the patella classifications 1208. In another example, training data 1232 may be generated pairing surgical plans 1210 for certain patella risk classifications with corresponding real-world patient outcomes (for instance, in a medical database of medical information 1230) indicating the accuracy of the generated surgical plans 1210, particularly for a specific patella classification 1208 combined with the associated patient information 1206.

Training data 1232 may be used to train computational model 1202 to accurately predict patella classifications 1208 for particular patella information 1204 and/or patient information 1206. Accordingly, computational model 1202 can “learn” via AI/ML processes combined with training data 1232 to correctly or substantially correctly diagnose a patient, provide a patella classification, and provide a surgical plan associated with the patient information (including the patella classification).

The foregoing description has broad application. While the present disclosure refers to certain any preceding or subsequent example, numerous modifications, alterations, and changes to the described examples are possible without departing from the sphere and scope of the present disclosure, as defined in the appended claim(s). Accordingly, it is intended that the present disclosure not be limited to the described examples. Rather these examples should be considered as illustrative and not restrictive in character. All changes and modifications that come within the spirit of the disclosure are to be considered within the scope of the disclosure. The present disclosure should be given the full scope defined by the language of the following claims, and equivalents thereof. The discussion of any example is meant only to be explanatory and is not intended to suggest that the scope of the disclosure, including the claims, is limited to these examples. In other words, while illustrative examples of the disclosure have been described in detail herein, it is to be understood that the described concepts may be otherwise variously embodied and employed, and that the appended claims are intended to be construed to include such variations, except as limited by the prior art. Unless otherwise defined, all technical terms used herein have the same meaning as commonly understood by one of ordinary skill in the art to which the disclosure belongs.

Directional terms such as top, bottom, superior, inferior, medial, lateral, anterior, posterior, proximal, distal, upper, lower, upward, downward, left, right, longitudinal, front, back, above, below, vertical, horizontal, radial, axial, clockwise, and counter-clockwise) and the like may have been used herein. Such directional references are only used for identification purposes to aid the reader's understanding of the present disclosure. For example, the term “distal” may refer to the end farthest away from the medical professional/operator when introducing a device into a patient, while the term “proximal” may refer to the end closest to the medical professional when introducing a device into a patient. Such directional references do not necessarily create limitations, particularly as to the position, orientation, or use of this disclosure. As such, directional references should not be limited to specific coordinate orientations, distances, or sizes, but are used to describe relative positions referencing particular examples. Such terms are not generally limiting to the scope of the claims made herein. Any example or feature of any section, portion, or any other component shown or particularly described in relation to any preceding or subsequent example of similar sections, portions, or components herein may be interchangeably applied to any other similar example or feature shown or described herein.

It should be understood that, as described herein, an “example” (such as illustrated in the accompanying Figures and/or “in any preceding or subsequent example”) may refer to an illustrative representation of an environment or article, component, system, process, and/or the like in which a disclosed concept or feature may be provided or embodied, or to the representation of a manner in which just the concept or feature may be provided or embodied. However, such illustrated examples are to be understood as examples (unless otherwise stated), and other manners of embodying the described concepts or features, such as may be understood by one of ordinary skill in the art upon learning the concepts or features from the present disclosure, are within the scope of the disclosure. Furthermore, references to “one example” of the present disclosure are not intended to be interpreted as excluding the existence of additional examples that also incorporate the recited features.

In addition, it will be appreciated that while the Figures may show one or more examples of concepts or features together in a single example of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one example can be used separately, or with another example to yield a still further example. Thus, it is intended that the present subject matter covers such modifications and variations as come within the scope of the appended claims and their equivalents.

As used herein, an element or step recited in the singular and proceeded with the word “a” or “an” should be understood as not excluding plural elements or steps, unless such exclusion is explicitly recited. It will be further understood that the terms “comprises” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

The phrases “at least one”, “one or more”, and “and/or”, as used herein, are open-ended expressions that are both conjunctive and disjunctive in operation. The terms “a” (or “an”), “one or more” and “at least one” can be used interchangeably herein.

Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

The foregoing discussion has been presented for purposes of illustration and description and is not intended to limit the disclosure to the form or forms disclosed herein. For example, various features of the disclosure are grouped together in one or more examples or configurations for the purpose of streamlining the disclosure. However, it should be understood that various features of the certain any preceding or subsequent example or configurations of the disclosure may be combined in alternate examples or configurations. Moreover, the following claims are hereby incorporated into this Detailed Description by this reference, with each claim standing on its own as a separate example of the present disclosure.

What is claimed is:

1. A computer-implemented method for planning a knee arthroplasty procedure, comprising, via a processor of a computing device:
receiving anatomical information of a knee joint, the anatomical information comprising patella information of a femur of a patient and femoral information of a femur of the patient,
generating three-dimensional (3D) models of at least one portion of the knee joint based on the anatomical information, the 3D models comprising a patella model and a femoral model;
characterizing a knee morphology of the knee joint, the knee morphology comprising a patella morphology of the patella and a femur morphology of the femur based on the 3D models; and
determining a patella risk classification based on the knee morphology, the patella risk classification indicating a risk of a patella complication of the knee arthroplasty procedure.

2. The computer-implemented method of claim 1, further comprising determining patella landmarks based on the patella model, wherein the knee morphology is determined based on the patella landmarks.

3. The computer-implemented method of claim 2, the patella landmarks comprising one or more of width points, height points, depth/thickness points, inferior articular point, articular perimeter points, posterior ridge points, centroid of the patella, or patella neutral point.

4. The computer-implemented method of claim 1, the femur of the knee joint comprising a femoral implant, wherein the femoral information comprises determined information for the femoral implant.

5. The computer-implemented method of claim 4, the femur morphology comprising an implant trochlear groove morphology.

6. The computer-implemented method of claim 5, the implant trochlear groove morphology determined based on a sulcus model associated with the femoral component.

7. The computer-implemented method of claim 1, the patella risk classification comprising at least one of a risk of maltracking of the patella, a risk of post-operative knee pain, or patellofemoral instability.

8. The computer-implemented method of claim 1, the patella risk classification based on one or more risk factors comprising at least one of a Wiberg index, patella size, pre-operative patellar shift, or femoral sulcus groove depth.

9. The computer-implemented method of claim 1, further comprising determining a surgical plan based on the patella risk classification, the surgical plan configured to address at least one complication associated with the patella risk classification.

10. The computer-implemented method of claim 9, the surgical plan comprising a determination of an optimal femoral component for the patella risk classification.

11. The computer-implemented method of claim 9, the surgical plan comprising a determination of an optimal femoral component placement for the patella risk classification.

12. The computer-implemented method of claim 9, the surgical plan comprising a determination of an optimal femoral preparation factor for the patella risk classification, the femoral preparation factor comprising a resection depth.

13. A computer-assisted surgical system, comprising:
at least one computing device, comprising:
a display device;
processing circuitry; and
a memory coupled to the processing circuitry, the memory comprising instructions that, when executed by the processing circuitry, cause the processing circuitry to:
receive anatomical information of a knee joint, the anatomical information comprising patella information of a femur of a patient and femoral information of a femur of the patient,
generate three-dimensional (3D) models of at least one portion of the knee joint based on the anatomical information, the 3D models comprising a patella model and a femoral model,
characterize a knee morphology of the knee joint, the knee morphology comprising a patella morphology of the patella and a femur morphology of the femur based on the 3D models, and
determine a patella risk classification based on the knee morphology, the patella risk classification indicating a risk of a patella complication of the knee arthroplasty procedure.

14. The system of claim 13, the instructions, when executed by the processing circuitry, to cause the processing circuitry to determine patella landmarks based on the patella model, wherein the knee morphology is determined based on the patella landmarks.

15. The system of claim 14, the patella landmarks comprising one or more of width points, height points, depth/thickness points, inferior articular point, articular perimeter points, posterior ridge points, centroid of the patella, or patella neutral point.

16. The system of claim 13, the femur of the knee joint comprising a femoral implant, wherein the femoral information comprises determined information for the femoral implant.

17. The system of claim 16, the femur morphology comprising an implant trochlear groove morphology.

18. The system of claim 17, the implant trochlear groove morphology determined based on a sulcus model associated with the femoral component.

19. The system of claim 13, the patella risk classification comprising at least one of a risk of maltracking of the patella, a risk of post-operative knee pain, or patellofemoral instability.

20. The system of claim 13, the patella risk classification based on one or more risk factors comprising at least one of a Wiberg index, patella size, pre-operative patellar shift, or femoral sulcus groove depth.

21. The system of claim 13, the instructions, when executed by the processing circuitry, to cause the processing circuitry to determine a surgical plan based on the patella risk classification, the surgical plan configured to address at least one complication associated with the patella risk classification.

22. The system of claim 21, the surgical plan comprising a determination of an optimal femoral component for the patella risk classification.

23. The system of claim 21, the surgical plan comprising a determination of an optimal femoral component placement for the patella risk classification.

24. The system of claim 21, the surgical plan comprising a determination of an optimal femoral preparation factor for the patella risk classification, the femoral preparation factor comprising a resection depth.
